(19) United States
(12) Patent Application Publication
(10) Pub. No.: US 2008/0280285 A1
(43) Pub. Date: Nov. 13, 2008

(54) Systems and Methods For Testing using Microfluidic Chips

(76) Inventors: Zongyuan G. Chen, Philadelphia, PA (US); Jing Wang, Upper Darby, PA (US); Michael Mauk, Wilmington, DE (US); Haim H. Bau, Swarthmore, PA (US); Daniel Malamud, New York, NY (US); William Abrams, Merion, PA (US); Raymond Niedbala, Alletown, PA (US); Hendrikus Johannes Tanke, Rijnsburg (NL); Paul L.A.M. Corstjens, Leiderdorp (NL)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/937,975

(22) Filed: Nov. 9, 2007

Related U.S. Application Data
(63) Continuation-in-part of application No. PCT/US2006/018481, filed on May 11, 2006.
Application No. PCT/US2006/018481 is a continuation-in-part of application No. PCT/US2006/018575, filed on May 11, 2006.
Application No. PCT/US2006/018575 is a continuation-in-part of application No. PCT/US2006/018534, filed on May 11, 2006.
(60) Provisional application No. 60/679,797, filed on May 11, 2005.
(60) Provisional application No. 60/679,798, filed on May 11, 2005.
(60) Provisional application No. 60/679,816, filed on May 11, 2005.
(60) Provisional application No. 60/679,797, filed on May 11, 2005.
(60) Provisional application No. 60/679,798, filed on May 11, 2005.
(60) Provisional application No. 60/679,816, filed on May 11, 2005.
(60) Provisional application No. 60/679,797, filed on May 11, 2005.
(60) Provisional application No. 60/679,798, filed on May 11, 2005.
(60) Provisional application No. 60/679,816, filed on May 11, 2005.

(51) Int. Cl. C12Q 1/70; B01J 19/00; C12M 1/40; C12Q 1/04; C12Q 1/68; C12P 19/34; C12M 1/34; G01N 30/00; C12M 1/33; G01N 33/536; G01N 33/00

(52) U.S. Cl. 435/5; 422/68.1; 435/289.1; 435/34; 435/6; 435/91.2; 422/70; 435/306.1; 436/536; 436/94; 436/86; 435/288.5; 435/287.2

(57) ABSTRACT

Disclosed are methods, devices and systems for biological and chemical sample processing using microfluidic chips. The disclosed microfluidic chips contain at least two detection zones for interacting with pre-selected RNA sequences, DNA sequences, antibodies, or antigens to determine their presence in the sample. Systems are also described comprising a cassette having at least one port and a sample inlet in fluid communication with a detection zone for interacting with pre-selected RNA sequences, DNA sequences, antibodies, or antigens, or mixtures thereof, if present, in a sample. Methods for concurrent testing of at least two of RNA, DNA, antibody, and antigen in a sample are also described, as are methods for testing for pre-selected pathogens and microfluidic methods.

### CROSS-REFERENCE TO RELATED APPLICATIONS

This application is a continuation-in-part of International Patent Application No. PCT/US2006/018481, filed May 11, 2006, which claims the benefit of priority to U.S. Provisional Application Ser. Nos. 60/679,797, filed May 11, 2005, 60/679,798, filed May 11, 2005, and 60/679,816, filed May 11, 2005, the disclosures of which are each incorporated herein by reference in their entireties. This application is also a continuation-in-part of International Patent Application No. PCT/US2006/018575, filed May 11, 2006, which claims the benefit of priority to U.S. Provisional Application Ser. Nos. 60/679,797, filed May 11, 2005, 60/679,798, filed May 11, 2005, and 60/679,816, filed May 11, 2005, the disclosures of which are each incorporated herein by reference in their entireties. This application is also a continuation-in-part of International Patent Application No. PCT/US2006/018534, filed May 11, 2006, which claims the benefit of priority to U.S. Provisional Application Ser. Nos. 60/679,797, filed May 11, 2005, 60/679,798, filed May 11, 2005, and 60/679,816, filed May 11, 2005, the disclosures of which are each incorporated herein by reference in their entireties.

### BACKGROUND

While clinical laboratories excel at detecting proteins and nucleotides, including genetic information, disease-causing agents, and indicators of disease or disorders, there is always a delay between sample collection and communication of the results of testing. In certain circumstances, such as a highly infectious outbreak or incident of bioterrorism, such a delay could be catastrophic. In such cases, facilitating testing where the sample is collected is a highly important goal.

Even under less dramatic circumstances where such testing is already a reality, improved testing is very desirable. For example, there are known tests used to detect HIV via the presence of antibodies to HIV. However, there is a six to twelve week period between HIV infection and measurable antibody response, during which time an infected individual can transmit the virus. This presents an unacceptable lag. Testing by clinical laboratories does not remedy the lag, because of the above-mentioned delay between acquiring a sample and informing the individual of the test results. Also, some patients never return after providing a sample, whereas if a sample could be diagnosed on-site with an immediate result, the individual could be counseled and appropriate therapy initiated.

Thus, testing devices and methods capable of detecting both the pathogen (via antigen and/or nucleic acid) and antibody to the pathogen are needed and would have tremendous impact on the diagnosis and monitoring of HIV. Of course, such testing devices and methods would be equally important for testing for other pathogens or diseases, or even pre-selected contaminants or pre-selected sequences, in fact, any nucleotide sequence, antigen, or antibody. Moreover, it is desirable that the testing devices and methods reduce costs. Finally, it is desirable that the testing be automated as far as possible to obtain the benefits of automation.

### SUMMARY OF THE INVENTION

The present invention relates to sample processing using a microfluidic chip. Microfluidic refers to the fact that a fluid is propelled through a system, allowing greater control. In some embodiments, the chips reduce processing time and materials. In some embodiments, the chips accommodate samples without pretreatment, or in a self-contained state to prevent cross-contamination. In some embodiments, the system allows for automatic processing. The present inventions also are suitable for use analyzing samples at the point of care, and in clinical laboratories, if the above-described delay is not a factor.

Accordingly, the present invention provides microfluidic chips comprising: a detection zone for interacting with pre-selected RNA sequences, DNA sequences, antibodies, or antigens, or mixtures thereof; at least one further detection zone for interacting pre-selected RNA sequences, DNA sequences, or antigens; and at least one flow path for contacting the detection zones with a sample.

The present invention also provides microfluidic chips, comprising: a detection zone for interacting with either pre-selected RNA sequences or pre-selected DNA sequences; and at least one further detection zone for interacting with pre-selected RNA sequences, DNA sequences, antibodies, or antigens.

The present invention also provides microfluidic chips, comprising at least one metering chamber.

The present invention also provides microfluidic chips, comprising: a PCR reaction chamber; and a phase change valve, a hydrogel valve, or mechanical valve.

The present invention also provides microfluidic chips, comprising: a detection zone for interacting with pre-selected RNA sequences, DNA sequences, antibodies, or antigens, or mixtures thereof; at least one further detection zone for interacting pre-selected RNA sequences, DNA sequences, antibodies, or antigens; wherein when the first detection zone is selected to interact with DNA sequences, the at least one further detection zone interacts with pre-selected RNA sequences, antibodies, or antigens, and wherein when the first detection zone is selected to interact with antigens, the at least one further detection zone interacts with pre-selected RNA sequences, DNA sequences, or antibodies; and at least one flow path for contacting the detection zones with a sample.

The present invention also provides microfluidic chips, comprising: two or more independent flow paths for separate assays wherein each flow path is comprised of sample processing steps for detecting one of predetermined sequences of DNA, predetermined sequences of RNA, antibody, or antigen.

The present invention also provides microfluidic chips, comprising: a diaphragm valve.

The present invention also relates to sample processing using a microfluidic cassette. Accordingly, the present invention provides methods for concurrent testing for at least two of RNA, DNA, antibody, and antigen in a sample, comprising: applying a portion of the sample to a detection zone disposed on a microfluidic cassette for interacting with pre-selected RNA sequences, DNA sequences, antibodies, or antigens, or mixtures thereof; and applying at least one further portion of the sample to at least one further detection zone disposed on the microfluidic cassette for interacting with pre-selected RNA sequences, DNA sequences, or antigens.

The present invention also provides methods for testing for the presence of a pre-selected pathogen in a sample, comprising: placing the sample in a microfluidic cassette; metering the sample; propelling the sample along a flow path in the cassette to a detection zone having at least one zone adapted to interact with the pre-selected pathogen; and detecting the presence or absence of interaction.

The present invention also provides methods of testing for pre-selected pathogens, comprising: placing a sample in a cassette; and propelling the sample through the cassette under pressure, wherein a portion of the sample is directed to a detection zone for interacting with pre-selected RNA sequences, DNA sequences, antibodies, or antigens known to be associated with a pre-selected pathogen.

The present invention also provides methods for testing for HIV in a sample, comprising: providing a microfluidic cassette having means for testing for RNA sequences associated with HIV and means for testing for antigens associated with HIV.

The present invention also provides methods for filling and emptying of a closed loop, comprising: providing an ice valve in the loop between an inlet and outlet; closing the valve to fill the loop; opening the valve to circulate fluid; and closing the valve to empty the loop out the outlet.

The present invention also provides methods for mixing fluids in a chamber without bubble formation, comprising: adding a fluid; freezing the fluid; adding at least one further fluid; and thawing the first fluid.

The present invention also provides methods for performing PCR in a chamber without bubble formation, comprising: providing a valve at each inlet and outlet of the chamber; and closing the valves.

The present invention also provides cassettes that reduce processing time and materials. In some embodiments, the cassettes accommodate samples without pretreatment, or in a self-contained state to prevent cross-contamination. In some embodiments, the system allows for automatic processing. The present inventions also are suitable for analyzing samples at the point of care, and in clinical laboratories, if the above-described delay is not a factor.

The present invention also provides systems comprising: a cassette having at least one port and a sample inlet in fluid communication with a detection zone for interacting with pre-selected RNA sequences, DNA sequences, antibodies, or antigens, or mixtures thereof, if present, in a sample; and a developer for engaging the port of the cassette, wherein the developer propels the sample from said inlet to said detection zone.

The present invention also provides systems comprising a cassette having at least one port and a sample inlet in fluid communication with a detection zone for interacting with pre-selected RNA sequences, DNA sequences, antibodies, or antigens, or mixtures thereof, if present, in a sample; a developer for engaging the port of the cassette, wherein the developer propels the sample from said inlet to said detection zone; and a detector for detecting the RNA, DNA, antibody, or antigen.

The general description and the following detailed description are exemplary and explanatory only and are not restrictive of the invention, as defined in the appended claims. Other aspects of the present invention will be apparent to those skilled in the art in view of the detailed description of the invention as provided herein.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale. In the drawings:

FIG. 1 is a schematic view of a developer according to the present invention.

FIG. 2 is a schematic view of the developer receiving a cassette.

FIG. 3 is a schematic view of an alternative developer.

FIG. 4 is a schematic view of a chip housed in the cassette.

FIG. 5 is a decision tree for valve control and/or reagent control.

FIG. 6 is a schematic of a portion of a cassette adapted to perform polymerase chain reaction (“PCR”) and valve settings.

FIG. 7 is a schematic of a system according to the present invention.

FIG. 8 is a schematic of a quick connection system for connecting of lines to the chip housed in the cassette.

FIG. 9 is a flow chart of testing according to the present invention.

FIG. 10 is a schematic view of a developer receiving a cassette.

FIG. 11 is a schematic view of a chip contained by the cassette.

FIG. 12 is a chart showing the various paths for DNA detection, antibody detection, antigen detection, and RNA detection.

FIG. 13 is a schematic and images of an ice valve.

FIG. 14 is a schematic of filling a chamber in a cassette.

FIGS. 15-17 are images of a portion of a cassette.

FIG. 18 is a chart showing detection by a cassette.

FIG. 19 is an image of a gel.

FIG. 20 is an image of a gel.

FIG. 21 is a schematic for metering in one embodiment.

FIG. 22 is a schematic view of a chip according to the present invention.

FIGS. 23A-B are a schematic view and image of an alternative embodiment of a chip.

FIG. 24 is a schematic view of a portion of a chip adapted to meter the sample.

FIG. 25 is a perspective view of a portion of a chip.

FIG. 26 is a top plan view of a portion of a chip adapted to perform polymerase chain reaction (“PCR”).

FIGS. 27A-B are images of a portion of a chip adapted to isolate nucleic acid.

FIG. 28 is an image of a portion of a chip adapted to perform PCR.

FIG. 29 is a chart showing the various paths for DNA detection, antibody detection, antigen detection, and RNA detection.

FIG. 30 is an image of a heater for the chip.

FIG. 31 is a schematic view and image of a check valve for the chip.

FIG. 32 is a schematic view and image of a mini-chip.

FIG. 33 is a schematic view and image of an alternative mini-chip.

FIG. 34 is a schematic view and image of a diaphragm valve for the chip.

FIG. 35 is a schematic view of a micropump for the chip.

FIG. 36 is a schematic view and image of a chip.

FIG. 37 is an image of a chip and housing.

It is understood that the figures are merely to illustrate certain features, and in no way limit the invention.

### DETAILED DESCRIPTION

The present invention may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

Systems of the present invention include a cassette having at least one port and a sample inlet in fluid communication with a detection zone for interacting with pre-selected RNA sequences, DNA sequences, antibodies, or antigens, or mixtures thereof, if present, in a sample; and a developer for engaging the port of the cassette, wherein the developer propels the sample from said inlet to said detection zone.

Referring to FIG. 1, a developer is shown having a chamber for receiving a cassette, such as a microfluidic chip containing cassette. In one embodiment, the chamber is refrigerated. Microfluidic refers to the fact that a fluid is propulsed through a system, allowing greater control. It is understood that the propulsion provided by the developer is hydraulic (either pressure or suction), pneumatic, electric, or magnetic. The developer supplies reagents that can be used in sample processing, sample treatment, or detection of interaction. In one embodiment, the developer dispenses a reagent for treating the sample. In some embodiments, the appropriate buffers and treatment fluids are pre-loaded on the cassette, and in some embodiments, some reagents are preloaded and some dispensed.

The developer also retains controls for controlling testing conditions and materials. Thus, in one embodiment, the developer provides electrical power. In another embodiment, the developer provides propulsion. The developer may also include a heater/cooler, such as a Peltier heater/cooler. In one embodiment, the cassette has a heater.

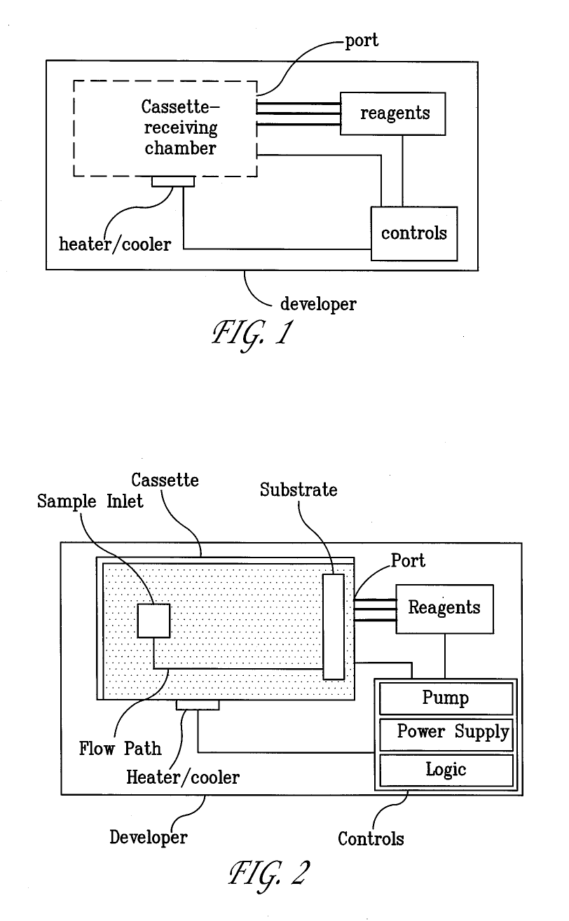

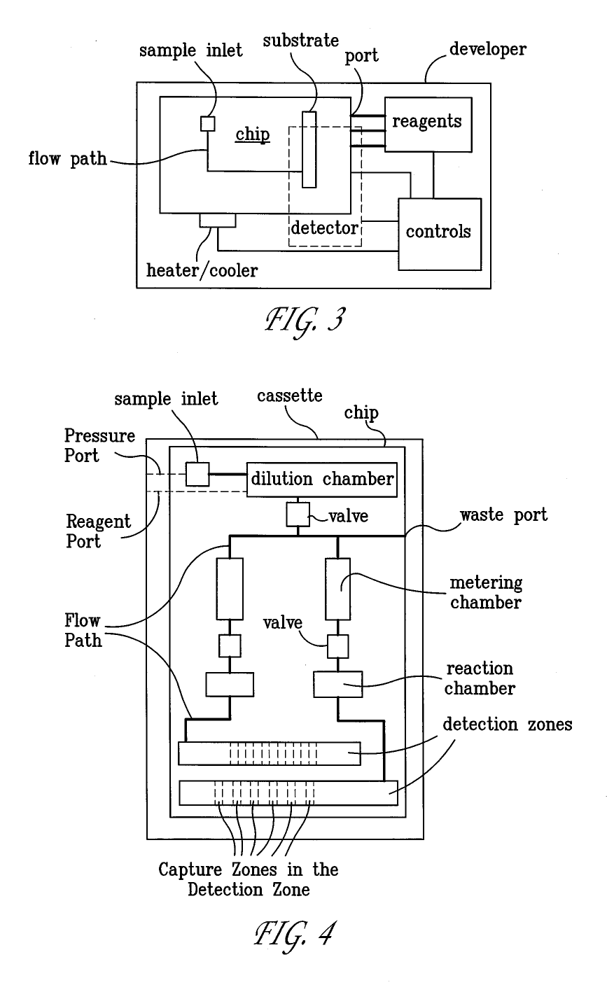

Turning to FIG. 2, the developer has received the cassette. It is understood that the cassette and developer are in fluid communication. A sample inlet is disposed in the cassette for introduction of a sample into the cassette. The sample can be any material that might contain RNA sequences, DNA sequences, antibodies, or antigens. Examples of samples include foodstuffs, water, saliva, blood, urine, fecal samples, lymph fluid, breast fluid, CSF, tears, nasal swabs, and surface swabs. In one embodiment, the cassette finds use in testing for pathogens, so the pre-selected sequences, antibodies, or antigens are those associated with at least one known pathogen. In another embodiment, the pre-selected sequences, antibodies, or antigens are those associated with more than one pathogen. Likewise, in one embodiment, the pre-selected sequences, antibodies, or antigens are those associated with at least one known disorder. In one embodiment, the cassette further comprises at least one further detection zone for interacting with RNA, DNA, or antigen, to allow parallel testing.

The detection zone is contacted with capture sequences that are pre-selected for the pathogen. In some embodiments, multiple pathogens are tested for by providing complementary sequences pre-selected for the pathogens. Likewise, in one embodiment, the at least one further detection zone is a chromatographic detection zone. In one embodiment, the detection zone comprises a polymeric material, such as a nitrocellulose strip. The detection zone is contacted with capture sequences that are pre-selected for the pathogen or compound of interest. In some embodiments, multiple pathogens are tested for by providing complementary sequences pre-selected for the pathogens. It is understood that a sample lacking the pathogen(s) or compound(s) of interest will not interact with the detection zone. If present, the interaction between sample and sequence(s) is detectable.

It is understood that the developer could receive more than one cassette to process at a time. It is also understood that the developer could process cassettes of varying types, limited only by the reagents stored (unless the cassettes were pre-loaded), for example, an HIV test cassette, a cancer detection cassette (p-54 mutation or protein indicator), and a cassette for determining presence of a hair color gene could all be processed by the developer. The developer may also dispense a reagent for diluting the sample. The dilution is optional, as it is understood that mixing the sample with buffer could serve a similar purpose.

A flow path extends between the sample inlet and the detection zone. In one embodiment, the first mentioned detection zone is a chromatographic detection zone. In one embodiment, the first mentioned detection zone is in a lateral flow format. In one embodiment, the detection zone comprises a polymeric material, such as a nitrocellulose strip. Likewise, in one embodiment, the at least one further detection zone is a chromatographic detection zone. In one embodiment, the detection zone is in a lateral flow format, and in one embodiment, the detection zone comprises a polymeric material, such as a nitrocellulose strip. In one embodiment, the cassette further comprises a plurality of detection zones, wherein each detection zone independently interacts with RNA, DNA, antigen, or antibody.

The first mentioned detection zone can have a pre-selected pattern of zones, each for interacting with a different sequence of RNA, DNA, antigen, or antibody. In one embodiment, the further detection zone has a pre-selected pattern of zones, each for interacting with a different sequence of RNA, DNA, antigen, or antibody.

The interaction is detectable in some embodiments, such as through the use of reporter particles. All known reporter particles are contemplated, for example, the reporter particles may be phosphor particles (such as Up-Converting Phosphor Technology (UPT) particles), fluorescing particles, hybridization sensors, or electrochemical sensors.

Additional microfluidic elements may also be included, for example, the cassette may further comprise a waste reservoir to limit contamination by the sample, or cross-contamination between cassettes, as well as keeping the bioactive waste on the cassette. Various valve types may also be included. It is understood that the valve could be any type of valve, including a phase change valve, piezo-electric valve, hydrogel valve, passive valve, check valve, or a membrane-based valve. In one embodiment, the valve is a phase change valve or a hydrogel valve.

The temperature-responsive hydrogel, poly(N-isopropylacrylamide), when saturated with an aqueous solution, undergoes a significant, reversible volumetric change when its temperature is increased from room temperature to above the phase transition temperature of about 32° C. The hydrogel can be embedded in polycarbonate plates prior to the thermal bonding of the plates. The exposure of the hydrogel to the thermal bonding temperatures does not have any apparent adverse effect on the gel. Moreover, one important advantage of the hydrogel valve is that when dry, it allows free passage of gases. In pneumatic systems, the dry hydrogel valve will allow the displacement of air from cavities and conduits upstream of an advancing liquid slug. Once the aqueous liquid arrives at the hydrogel's location, it will saturate and swell the gel, blocking the flow passage. Thus, the valve is self-actuated. The valve can be opened by heating the hydrogel to above its transition temperature. The hydrogel proved to be biocompatible in our testing and did not to hinder PCR. Moreover, the hydrogel valves did not appear to absorb significant quantities of DNA and enzymes suspended in PCR buffer.

Ice valves take advantage of the phase change of the working liquid itself—the freezing and melting of a portion of a liquid slug—to non-invasively close and open flow passages. An ice valve is electronically-addressable, does not require any moving parts, introduces only minimal dead volume, is leakage and contamination free, and is biocompatible. Moreover, in certain cases, the valve can operate in a self-actuated mode, alleviating the need for a sensor to determine the appropriate actuation time. For example, in a pneumatically driven system, the precooled conduit section would allow the free passage of air prior to the arrival of the liquid slug and would seal at the desired time when the slug arrives at the valve location.

The developer may further include has means for controlling the valve. Suitable means for controlling the valve includes a heater/cooler, optionally controlled by logic.

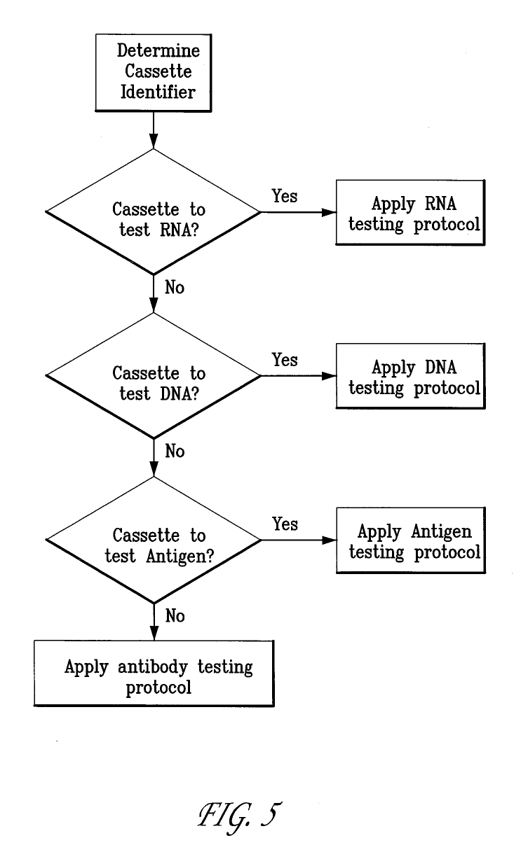

Referring to FIG. 3, the developer may optionally have a detector for detecting interaction. Alternatively, the detector may be a stand alone detector, to allow the developer to remain dedicated to developing cassettes, allowing faster process times. Thus, in one embodiment, the system further comprises a detector for detecting the RNA, DNA, antibody, or antigen. The present invention, in one embodiment, provides a system, comprising a cassette having at least one port and a sample inlet in fluid communication with a detection zone for interacting with pre-selected RNA sequences, DNA sequences, antibodies, or antigens, or mixtures thereof, if present, in a sample; a developer for engaging the port of the cassette, wherein the developer propels the sample from said inlet to said detection zone; and a detector for detecting the RNA, DNA, antibody, or antigen. In one embodiment, the detector is a UPT detector.

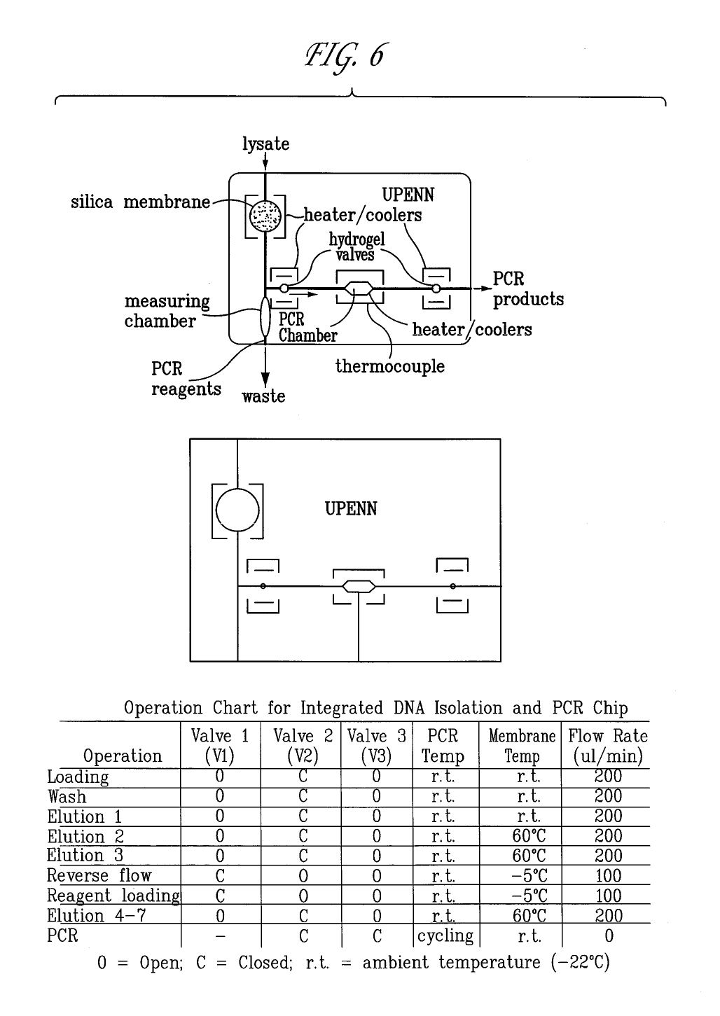

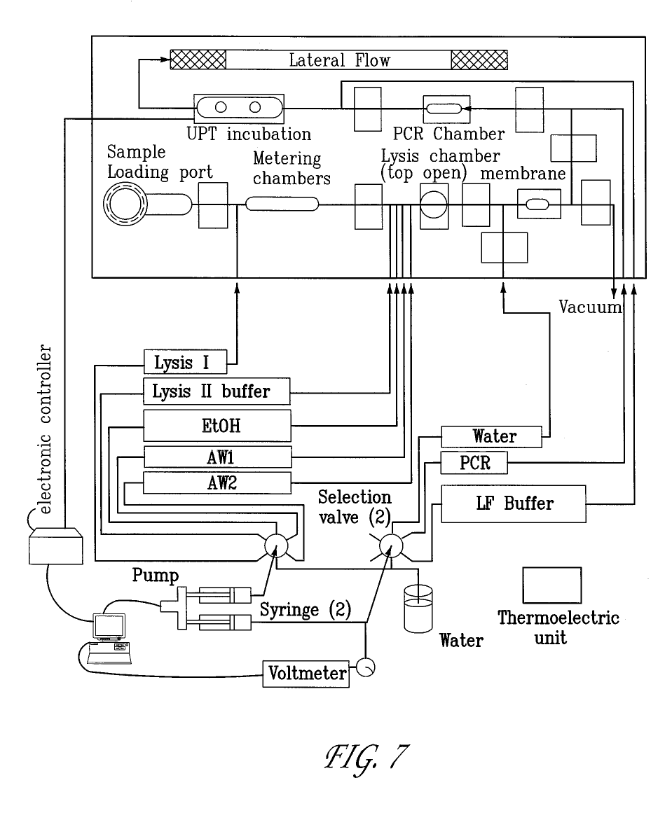

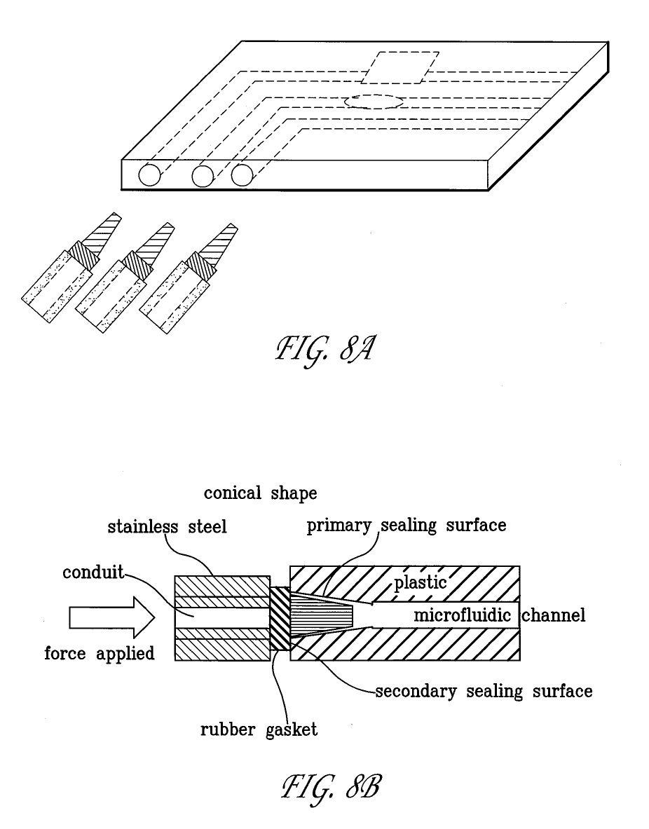

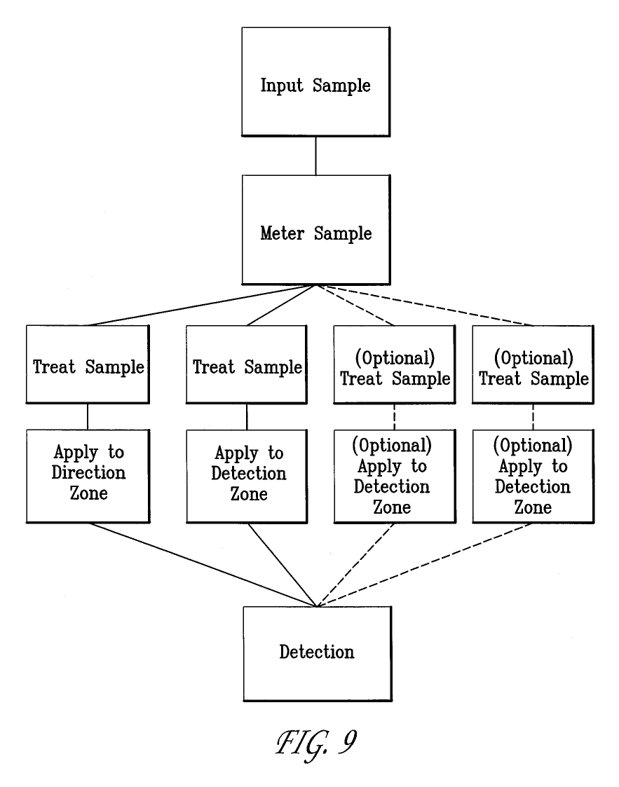

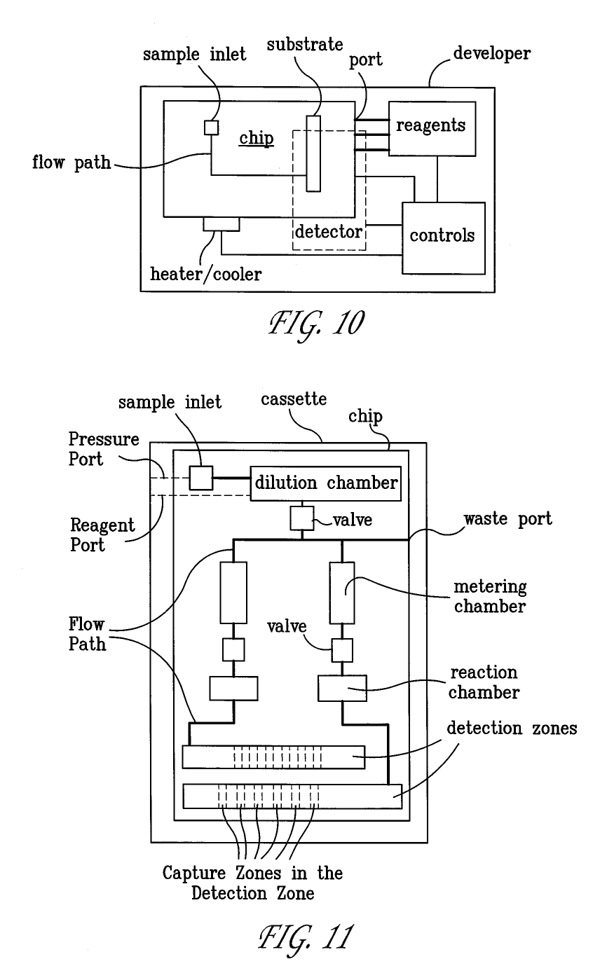

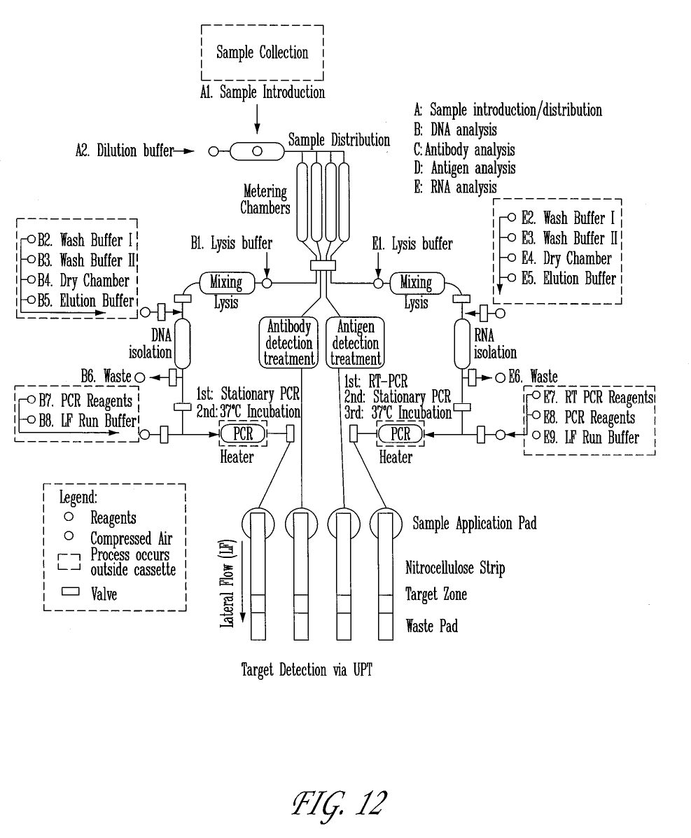

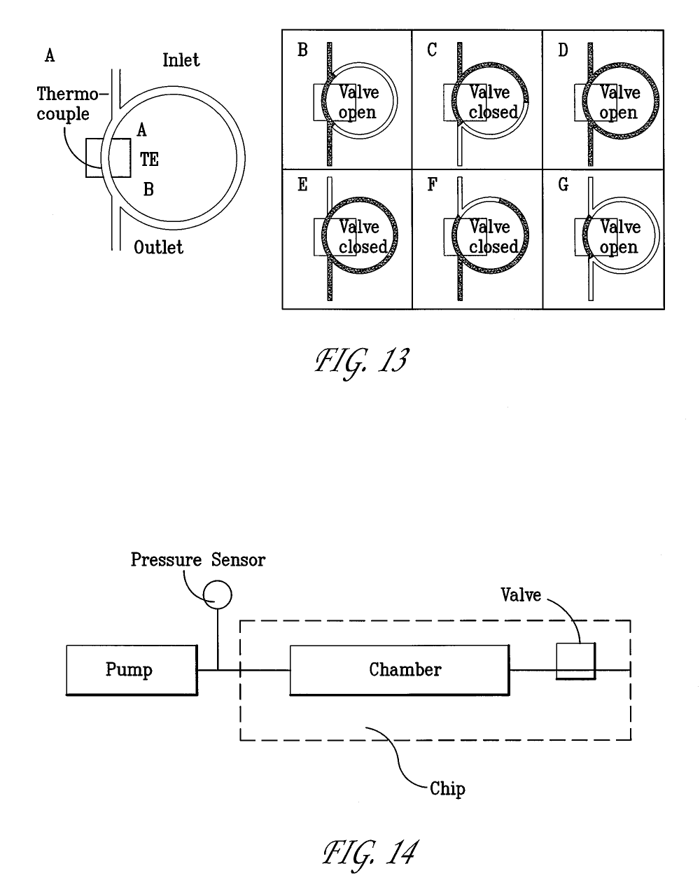

Turning to FIG. 4, an exemplary chip housed by the cassette is depicted. The chips can be preloaded and stored.

Referring back to FIG. 2, optionally, the cassette bears an identifier to indicate the type of pathogen(s) to be detected with the cassette. In one embodiment, the identifier is a barcode (either mechanical or optical), RFID tag, or mechanical change in the surface of the cassette. It can be appreciated that the identifier could be associated with certain information that is known at the time that the cassette is fabricated, for example, how many detection zones are on the cassette, what disease-causing agents or indicators of disease are being tested for, and whether each detection zone requires is detecting RNA, DNA, antibody, or antigen. The identifier could also be associated with certain information at the time of testing, for example, a unique patient identifier, sample type, and patient factors (age, health, suspected disorder). In one embodiment, the identifier dictates the sequence of operations to the developer in order to process the cassette.

Turning to FIG. 5, the developer can use the identifier to determine the appropriate analysis path. The analysis path for the detection of DNA includes the following main steps: pathogen lysis; DNA isolation and purification; PCR; isolation of the amplified DNA; mixing and incubation with target specific reporter particles; and capture of the labeled amplicon on a lateral flow strip. The analysis path for the detection of RNA comprises: cell lysis; RNA isolation and purification; Reverse Transcription PCR; isolation of the amplified DNA; mixing and incubation with target specific reporter particles; and capture of the labeled amplicons on a lateral flow strip. The analysis path for the detection of human antibodies to select pathogens comprises: dilution of sample; mixing and incubation with target specific reporter particles; capture on a lateral flow strip. The analysis path for the detection of pathogen antigens comprises dilution; solubilization or release of antigen; mixing and incubation with target specific reporter particles; and capture of labeled antigen on a lateral flow strip. Of course, the analysis paths described above focused on the lateral flow format. The invention also includes consecutive flow assays for the detection of antibodies. In the case of the consecutive flow assay, the analysis path will comprise: dilution, capture/enrichment of specific antibodies on a lateral flow strip; wash step to remove unbound antibodies; and detection by flowing reporter particles over the lateral flow strip.

Thus, in one embodiment, the developer provides treating reagent directed to RNA isolation and amplification. In another embodiment, the developer provides treating reagent directed to DNA isolation and amplification. In another embodiment, the developer provides treating reagent directed to antibody detection. In another embodiment, the developer provides treating reagent directed to antigen detection.

Likewise, unless the reagent has been pre-loaded, the developer dispenses a reagent for labeling the interacted RNA, DNA, antibody, or antigen with a reporter particle.

Turning to FIG. 6, when the reaction chamber is a PCR chamber, the format can be stationary (sample held in a chamber that is alternately heated and cooled, continuous flow through (sample propelled through a serpentine channel passing through a plurality of heating zones), pneumatic oscillatory (sample propelled back and forth through a conduit passing through a plurality of heating zones), self actuated (sample propelled through a closed loop containing a plurality of heating zones), electrokinetic (sample propelled by an electric field), or magneto-hydrodynamically (MHD)-driven (flow induced by electric current in the presence of a magnetic field). The illustrated portion of a cassette in FIG. 6 is adapted to perform PCR. The portion receives cells, lyses them, isolates nucleotide sequences, then amplifies them via PCR. The developer has logic to control the valve settings as listed, thereby allowing for proper treatment.

FIG. 7 illustrates a schematic of the system in one embodiment of the present invention, including a chip and developer components.

Referring now to FIG. 8, a schematic of a quick connection system for connecting of lines to a chip inside the cassette is shown. The connection between external fluidic (e.g., vacuum, hydraulic, or pneumatic pressure, sample, reagent and buffer supplies) lines and the cassette is a challenge. In one embodiment, a relatively-soft material such as plastic is used. Application of a moderate force on a male end against a female end generates a subtle deformation around the conical-shaped interface, thus forming the primary sealing surface. A gasket functions as a secondary sealing surface. This dual sealing-surface approach secures a satisfactory, quick, leak-free connection between off-chip fluidic lines and the chip. It is understood that the depicted stainless steel, rubber, and plastic materials are exemplary not intended to limit the invention.

Methods for testing for a pre-selected pathogen in a sample are also provided in the present invention. Embodiments of these methods include placing the sample in a microfluidic cassette; propelling the sample along a flow path in the cassette to a detection zone having at least one zone adapted to interact with the pre-selected pathogen; and detecting the presence or absence of interaction. In one embodiment, there is a pre-selected pattern of zones on the detection zone, each for interacting with a different sequence. In one embodiment, the method further comprises applying a portion of the sample to a pre-selected pattern of zones on at least one further detection zone, each zone for interacting with a different sequence of RNA, DNA, antigen, or antibody.

Turning to FIG. 9, a method of testing is shown, comprising obtaining a sample, metering the sample, treating portions of the sample, applying the portions to a detection zone, and detecting interactions that would indicate the presence of a disease or a disease-indicator.

Referring to FIG. 10, a developer is shown having a chamber for receiving a cassette, such as a microfluidic chip containing cassette. In one embodiment, the chamber is temperature controlled. It is understood that the propulsion provided by the developer is hydraulic (either pressure or suction), pneumatic, electric, or magnetic. In FIG. 11, an exemplary microfluidic chip contained in the cassette is depicted. In some embodiments, the microfluidic flow path channels have a diameter of about 1 mm or less.

In some embodiments, the developer supplies reagents that can be used in sample processing, sample treatment, or detection of interaction. In one embodiment, the developer dispenses a reagent for treating the sample. In some embodiments, the appropriate buffers and treatment fluids are pre-loaded on the cassette, and in some embodiments, some reagents are preloaded and some dispensed. The developer also retains controls for controlling testing conditions and materials. Thus, in one embodiment, the developer provides electrical power. In another embodiment, the developer provides propulsion. In one embodiment, the developer includes a heater/cooler, such as a Peltier heater/cooler. In one embodiment, the cassette has a heater.

It is understood that the cassette and developer are in fluid communication. A sample inlet is disposed in the cassette for introduction of a sample into the cassette. The sample can be any material that might contain RNA sequences, DNA sequences, antibodies, or antigens. Examples of samples include foodstuffs, water, saliva, blood, urine, fecal samples, lymph fluid, breast fluid, CSF, tears, nasal swabs, and surface swabs. In one embodiment, the cassette finds use in testing for pathogens, so the pre-selected sequences, antibodies, or antigens are those associated with at least one known pathogen. In another embodiment, the pre-selected sequences, antibodies, or antigens are those associated with more than one pathogen. Likewise, in one embodiment, the pre-selected sequences, antibodies, or antigens are those associated with at least one known disorder. In one embodiment, the cassette further comprises at least one further detection zone for interacting with RNA, DNA, or antigen, to allow parallel testing.

The detection zone is contacted with capture sequences that are pre-selected for the pathogen. In some embodiments, multiple pathogens are tested for by providing complementary sequences pre-selected for the pathogens. Likewise, in one embodiment, the at least one further detection zone is a chromatographic detection zone. In one embodiment, the detection zone comprises a polymeric material such as a nitrocellulose strip. The detection zone is contacted with capture sequences that are pre-selected for the pathogen or compound of interest. In some embodiments, multiple pathogens are tested for by providing complementary sequences pre-selected for the pathogens. It is understood that a sample lacking the pathogen(s) or compound(s) of interest will not interact with the detection zone. If present, the interaction between sample and sequence (s) is detectable.

It is understood that the developer could receive more than one cassette to process at a time. It is also understood that the developer could process cassettes of varying types, limited only by the reagents stored (unless the cassettes were pre-loaded), for example, an HIV test cassette, a cancer detection cassette (p-54 mutation or protein indicator), and a cassette for determining presence of a hair color gene could all be processed by the developer.

In one embodiment, the developer dispenses a reagent for diluting the sample. The dilution is optional, as it is understood that mixing the sample with buffer could serve a similar purpose. A flow path extends between the sample inlet and the detection zone. In one embodiment, the first mentioned detection zone is a chromatographic detection zone. In one embodiment, the first mentioned detection zone is in a lateral flow format. In one embodiment, the detection zone is a polymeric material such as a nitrocellulose strip. Likewise, in one embodiment, the at least one further detection zone is a chromatographic detection zone. In one embodiment, the detection zone is in a lateral flow format, and in one embodiment, the detection zone is a polymeric material such as a nitrocellulose strip. In one embodiment, the cassette further comprises a plurality of detection zones, wherein each detection zone independently interacts with RNA, DNA, antigen, or antibody. In one embodiment, the first mentioned detection zone has a pre-selected pattern of zones, each for interacting with a different sequence of RNA, DNA, antigen, or antibody. In one embodiment, the further detection zone has a pre-selected pattern of zones, each for interacting with a different sequence of RNA, DNA, antigen, or antibody. In some embodiments, the interaction is detectable, such as through reporter particles. All known reporter particles are contemplated, for example, the reporter particles may be phosphor particles (such as Up-Converting Phosphor Technology (UPT) particles), fluorescing particles, hybridization sensors, or electrochemical sensors.

In one embodiment, the cassette further comprises a waste reservoir to limit contamination by the sample, or cross-contamination between cassettes, as well as keeping the bioactive waste on the chip.

Various valve types are contemplated. It is understood that the valve could be any type of valve, including a phase change valve, piezo-electric valve, hydrogel valve, passive valve, check valve, or a membrane-based valve. In one embodiment, the valve is a phase change valve or a hydrogel valve. The temperature-responsive hydrogel, poly(N-isopropylacrylamide), when saturated with an aqueous solution, undergoes a significant, reversible volumetric change when its temperature is increased from room temperature to above the phase transition temperature of about 32° C. The hydrogel can be embedded in polycarbonate plates prior to the thermal bonding of the plates. The exposure of the hydrogel to the thermal bonding temperatures does not have any apparent adverse effect on the gel. Moreover, one important advantage of the hydrogel valve is that when dry, it allows free passage of gases. In pneumatic systems, the dry hydrogel valve will allow the displacement of air from cavities and conduits upstream of an advancing liquid slug. Once the aqueous liquid arrives at the hydrogel's location, it will saturate and swell the gel, blocking the flow passage. Thus, the valve is self-actuated. The valve can be opened by heating the hydrogel to above its transition temperature. The hydrogel proved to be biocompatible in our testing and did not to hinder PCR. Moreover, the hydrogel valves did not appear to absorb significant quantities of DNA and enzymes suspended in PCR buffer. Ice valves take advantage of the phase change of the working liquid itself—the freezing and melting of a portion of a liquid slug—to non-invasively close and open flow passages. An ice valve is electronically-addressable, does not require any moving parts, introduces only minimal dead volume, is leakage and contamination free, and is biocompatible. Moreover, in certain cases, the valve can operate in a self-actuated mode, alleviating the need for a sensor to determine the appropriate actuation time. For example, in a pneumatically driven system, the precooled conduit section would allow the free passage of air prior to the arrival of the liquid slug and would seal at the desired time when the slug arrives at the valve location. In one embodiment, the developer has means for controlling the valve. In one embodiment, the means is a heater/cooler, optionally controlled by logic.

Referring to FIG. 10, the developer may optionally have a detector for detecting interaction. Alternatively, the detector may be a stand alone detector, to allow the developer to remain dedicated to developing cassettes, allowing faster process times. Thus, in one embodiment, the system further comprises a detector for detecting the RNA, DNA, antibody, or antigen. The present invention, in one embodiment, provides a system, comprising a cassette having at least one port and a sample inlet in fluid communication with a detection zone for interacting with pre-selected RNA sequences, DNA sequences, antibodies, or antigens, or mixtures thereof, if present, in a sample; a developer for engaging the port of the cassette, wherein the developer propels the sample from said inlet to said detection zone; and a detector for detecting the RNA, DNA, antibody, or antigen. In one embodiment, the detector is a UPT detector.

Optionally, the cassette bears an identifier to indicate the type of pathogen(s) to be detected with the cassette. In one embodiment, the identifier is a barcode (either mechanical or optical), RFID tag, or mechanical change in the surface of the cassette. It can be appreciated that the identifier could be associated with certain information that is known at the time that the cassette is fabricated, for example, how many detection zones are on the cassette, what disease-causing agents or indicators of disease are being tested for, and whether each detection zone requires is detecting RNA, DNA, antibody, or antigen. The identifier could also be associated with certain information at the time of testing, for example, a unique patient identifier, sample type, and patient factors (age, health, suspected disorder).

Turning to FIG. 12, the developer can use the identifier to determine the appropriate analysis path. The analysis path for the detection of DNA will include the following main steps: pathogen lysis; DNA isolation and purification; PCR; isolation of the amplified DNA; mixing and incubation with target specific reporter particles; and capture of the labeled amplicon on a lateral flow strip. The analysis path for the detection of RNA comprises: cell lysis; RNA isolation and purification; Reverse Transcription PCR; isolation of the amplified DNA; mixing and incubation with target specific reporter particles; and capture of the labeled amplicons on a lateral flow strip. The analysis path for the detection of human antibodies to select pathogens comprises: dilution of sample; mixing and incubation with target specific reporter particles; capture on a lateral flow strip. The analysis path for the detection of pathogen antigens comprises dilution; solubilization or release of antigen; mixing and incubation with target specific reporter particles; and capture of labeled antigen on a lateral flow strip. Of course, the analysis paths described above focused on the lateral flow format. The invention also includes consecutive flow assays for the detection of antibodies. In the case of the consecutive flow assay, the analysis path will comprise: dilution, capture/enrichment of specific antibodies on a lateral flow strip; wash step to remove unbound antibodies; and detection by flowing reporter particles over the lateral flow strip.

Thus, in one embodiment, the developer provides treating reagent directed to RNA isolation and amplification. In another embodiment, the developer provides treating reagent directed to DNA isolation and amplification. In another embodiment, the developer provides treating reagent directed to antibody detection. In another embodiment, the developer provides treating reagent directed to antigen detection. Likewise, unless the reagent has been pre-loaded, the developer dispenses a reagent for labeling the interacted RNA, DNA, antibody, or antigen with a reporter particle.

Methods for concurrent testing of at least two of RNA, DNA, antibody, and antigen in a sample inlcude applying a portion of the sample to a detection zone disposed on a microfluidic cassette for interacting with pre-selected RNA sequences, DNA sequences, antibodies, or antigens, or mixtures thereof; and applying at least one further portion of the sample to at least one further detection zone disposed on the microfluidic cassette for interacting with pre-selected RNA sequences, DNA sequences, or antigens. These methods may further comprise applying a portion of the sample to another detection zone, wherein the detection zone interacts with RNA, DNA, antigen, or antibody. In one embodiment, the method further comprises detecting the interaction. In one embodiment, the interaction is detected using UPT particles, fluorescing particles, hybridization sensors, or electrochemical sensors.

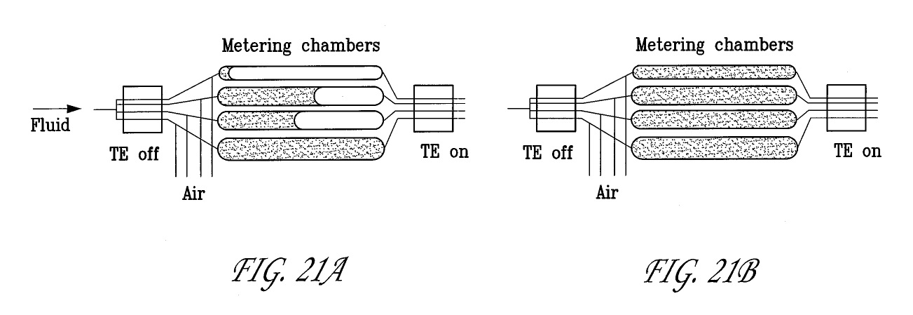

Methods for testing for pre-selected pathogens include placing a sample in a cassette; and propelling the sample through the cassette under pressure, wherein a portion of the sample is directed to a detection zone for interacting with pre-selected RNA sequences, DNA sequences, antibodies, or antigens known to be associated with a pre-selected pathogen. These methods may further comprise controlling movement of the sample with a valve disposed in the cassette. In one embodiment, the method further comprises diluting the sample. These methods may further comprise metering the sample. In one embodiment, FIG. 21 shows schematic description of the displacement process. (A) A sample flows into the metering chambers and displaces air out of the downstream phase change valve; (B) The sample fills all the metering chambers and freezes at the phase change valve.

These methods may further comprise treating the sample. In one embodiment, the method further comprises lysing cells in the sample. In one embodiment, the method further comprises isolating RNA or DNA in the sample. In one embodiment, the RNA or DNA are attached to a solid support. For example, these methods may further comprise amplifying RNA or DNA in the sample. In one embodiment, the RNA or DNA is amplified using PCR.

The methods described herein may further comprises detecting the interaction by attachment of a label. Suitable labels include UPT particles or fluorescing particles.

Methods for testing for HIV in a sample comprise providing a microfluidic cassette having means for testing for RNA sequences associated with HIV and means for testing for antigens associated with HIV.

When the reaction chamber is a PCR chamber, the format can be stationary (sample held in a chamber that is alternately heated and cooled, continuous flow through (sample propelled through a serpentine channel passing through a plurality of heating zones), pneumatic oscillatory (sample propelled back and forth through a conduit passing through a plurality of heating zones), self actuated (sample propelled through a closed loop containing a plurality of heating zones), electrokinetic (sample propelled by an electric field), or magneto-hydrodynamically (MHD)-driven (flow induced by electric current in the presence of a magnetic field). The developer has logic to control the valve settings as listed, thereby allowing for proper treatment.

Referring to FIG. 13, in certain applications such as MHD driven circular chromatography, MHD-driven PCR, MHD stirrer, and self-actuated flow-cycling PCR, it is desirable to operate in a closed loop. Ice valving provides a unique solution to the filling and emptying of a closed loop without any influence on the flow pattern along the whole loop. The filling of the closed loop without creating gas bubbles can be easily carried out. In FIG. 13, a closed loop is depicted equipped with an ice valve to aid in the filling and withdrawal of a liquid sample. The loop is connected to an inlet conduit and an exit conduit at points A and B, respectively. The inlet and exit conduits divide the loop into a long arc segment and a short arc segment. The thermoelectric unit is installed to cool part of the shorter arc segment between the inlet and outlet conduits (A). (B) provides a photograph of the loop equipped with the thermoelectric unit and fabricated with polycarbonate. The (B)-(G) depict the sequence of steps needed to fill (first row) and empty (second row) the loop. Initially, the valve is open. A liquid slug enters through the inlet conduit and fills the short arc segment between the inlet and the exit conduits (B). This is the path of least resistance to the flow. Next, a portion of the liquid slug is frozen (the valve closes), and the slug flows through the longer (right) arc (C) until the loop fills entirely with liquid (D). At this point in time, the PC valve opens, and the two other valves (not shown here) upstream and downstream of the loop are closed. The sample can now circulate around the loop as many times as desired. To withdraw the sample from the loop, the upstream and downstream valves (not shown) are opened and the PC valve along the short segment of the loop is closed (E). A gas stream delivered through the inlet conduit (F) displaces the sample (G).

Thus, in another embodiment of the present invention, a method is provided for filling and emptying of a closed loop, comprising providing an ice valve in the loop between an inlet and outlet; closing the valve to fill the loop; opening the valve to circulate fluid; and closing the valve to empty the loop out the outlet. These methods can also mix fluids in a chamber without bubble formation by adding a fluid; freezing the fluid; adding at least one further fluid; and thawing the first fluid.

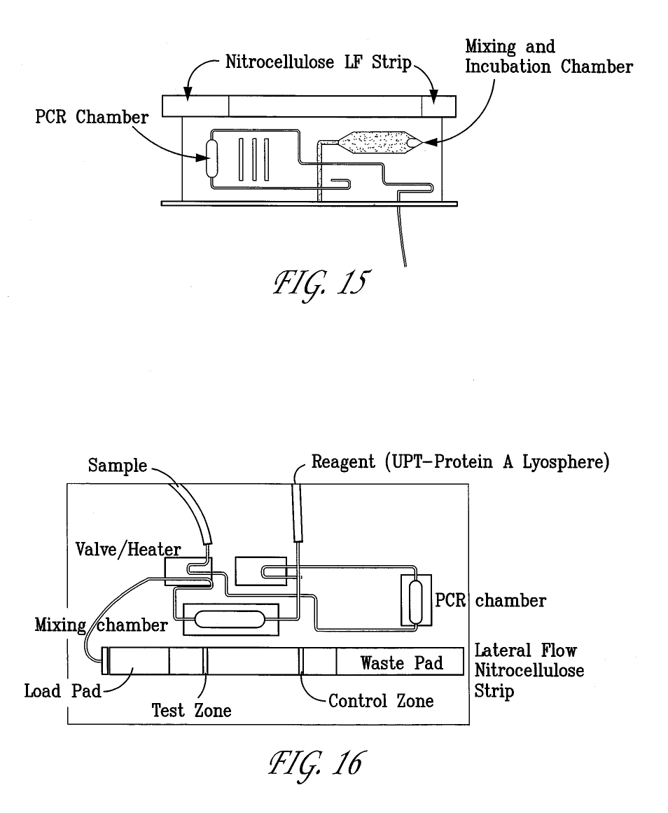

Referring to FIG. 14, in another embodiment of the present invention, a method is provided for automating flow control in a microfluidic cassette without the need for a sensor on the cassette, comprising propelling a fluid; freezing the fluid at the valve location via self-actuation (ice or hydrogel valve); external pressure sensor detecting the pressure increase; and stopping the fluid propelling.

In another embodiment of the present invention, a method is provided for performing PCR in a chamber without bubble formation, comprising providing a valve at each inlet and outlet of the chamber; and closing the valves. One mode of achieving cassette-based PCR is to hold the reagents in a chamber while cycling the chamber temperature (stationary PCR). One of the problems often experienced with stationary PCR microreactors is bubble formation. The bubbles are undesirable, as they may expel the reagents from the PCR chamber, thereby reducing the amplification efficiency. One way to minimize or eliminate the bubble formation is to pressurize the PCR chamber by sealing it. The PCR mixture is driven into the reaction chamber through the inlet phase change (PC) valve. During this process, the inlet valve is maintained at room temperature, allowing unhindered passage of the liquid. The liquid fills the PCR chamber, displacing the air through the pre-cooled exit valve. Once the air has been displaced out of the chamber and the liquid arrives at the exit valve's location, it freezes and blocks the passage. Subsequently, the inlet PC valve is closed. Once both the upstream and downstream valves are closed, the temperature of the PCR reactor is cycled according to standard protocols. The subsequent increase in pressure suppresses bubble formation.

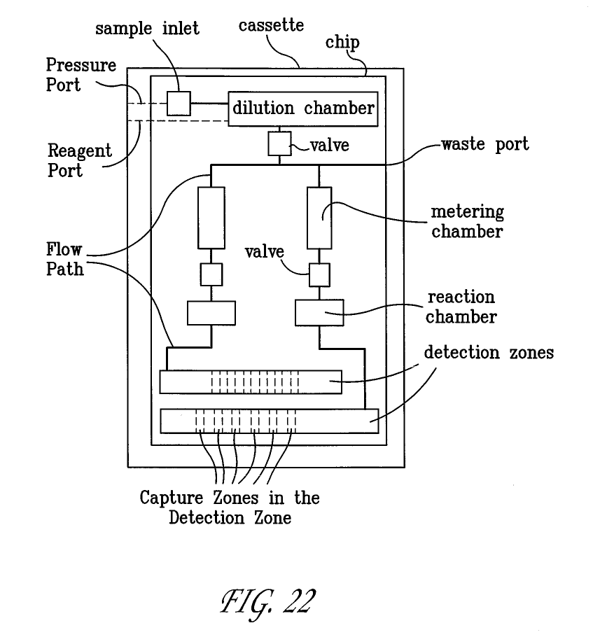

In yet other embodiments, the present invention provides chips, comprising a detection zone for interacting with pre-selected RNA sequences, DNA sequences, antibodies, or antigens, or mixtures thereof; at least one further detection zone for interacting with pre-selected RNA sequences, DNA sequences, or antigens; and at least one flow path for contacting the detection zones with a sample. Turning to FIG. 22, an exemplary chip is depicted. In one embodiment, the chip is a microfluidic chip. The chip can be formed from a variety of materials, including, for example, polycarbonate. In one embodiment, all steps from sample introduction to detection is integrated in a single chip. In one embodiment, the chip is formed from laminated polycarbonate sheets made from injection or hot embossing.

A sample inlet is disposed in the chip for introduction of a sample into the chip. The sample can be any material that might contain RNA sequences, DNA sequences, antibodies, or antigens. Examples of samples include foodstuffs, water, saliva, blood, urine, fecal samples, lymph fluid, breast fluid, CSF, tears, nasal swabs, and surface swabs. In one embodiment, the chip finds use in testing for pathogens, so the pre-selected sequences, antibodies, or antigens are those associated with at least one known pathogen. In another embodiment, the pre-selected sequences, antibodies, or antigens are those associated with more than one pathogen. Likewise, in one embodiment, the pre-selected sequences, antibodies, or antigens are those associated with at least one known disorder. An optional dilution chamber is shown in the chip, however, it is understood that mixing the sample with buffer could serve a similar purpose.

A flow path extends between the sample inlet and the detection zone. In one embodiment, the first mentioned detection zone is a chromatographic detection zone. In one embodiment, the first mentioned detection zone is in a lateral flow format. In one embodiment, the detection zone is a polymeric material such as a nitrocellulose strip. In one embodiment, the detection zone is an array of pillars that facilitate capillary propulsion. In one embodiment, the detection zone is an array of grooves. Likewise, in one embodiment, the at least one further detection zone is a chromatographic detection zone. In one embodiment, the detection zone is in a lateral flow format, and in one embodiment, the detection zone is a polymeric material such as a nitrocellulose strip. In one embodiment, the detection zone is an array of pillars that facilitate capillary propulsion. In one embodiment, the chip further comprises a plurality of detection zones, wherein each detection zone independently interacts with RNA, DNA, antigen, or antibody.

In one embodiment, the first mentioned detection zone has a pre-selected pattern of zones, each for interacting with a different sequence of RNA, DNA, antigen, or antibody.

In one embodiment, the further detection zone has a pre-selected pattern of zones, each for interacting with a different sequence of RNA, DNA, antigen, or antibody.

In some embodiments, the interaction is detectable, such as through reporter particles. All known reporter particles are contemplated, for example, the reporter particles may be phosphor particles (such as Up-Converting Phosphor Technology (UPT) particles), fluorescing particles, magnetic particles, particle arrays, hybridization sensors, or electrochemical sensors.

Optionally, the chip bears an identifier to indicate the type of pathogen(s) to be detected with the chip. In one embodiment, the identifier is a barcode (either manual or optical), RFID tag, or mechanical change in the surface of the chip.

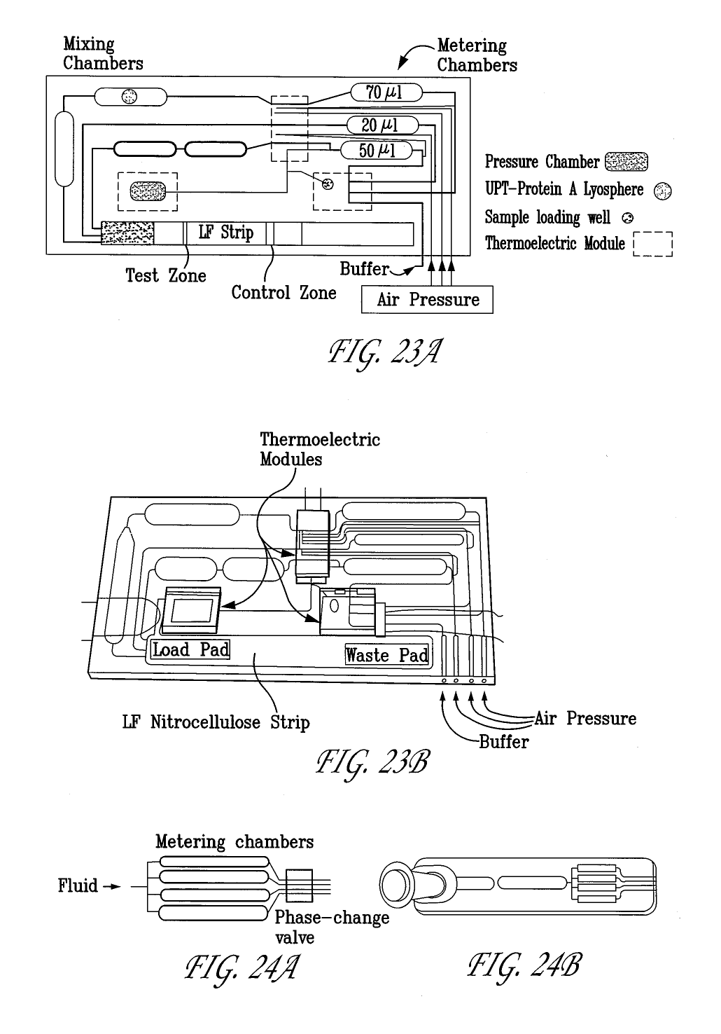

Referring now to FIGS. 22-25 in yet another embodiment of the present invention, a microfluidic chip is provided, comprising at least one metering chamber. A manifold that divides the sample into a plurality of metering chambers of pre-selected volumes is shown. As the sample enters through the inlet conduits, it fills the metering chambers, and displaces air through the outlet conduits. The chamber that offers the smallest hydraulic resistance fills first. Once the liquid arrives at the valve location, the valve closes and does not allow further liquid flow. In one embodiment, the chip further comprises a waste reservoir to limit contamination by the sample, or cross-contamination between chips, as well as keeping the bioactive waste on the chip.

Various valve types are contemplated. It is understood that the valve could be any type of valve, including a phase change valve, piezo-electric valve, hydrogel valve, passive valve, check valve, or a membrane-based valve. In one embodiment, the valve is a phase change valve or a hydrogel valve. In one embodiment, a phase-change valve is used to achieve metering, switching of flow, and sealing of a chamber.

The temperature-responsive hydrogel, poly(N-isopropylacrylamide), when saturated with an aqueous solution, undergoes a significant, reversible volumetric change when its temperature is increased from room temperature to above the phase transition temperature of about 32° C. The hydrogel can be embedded in polycarbonate plates prior to the thermal bonding of the plates. The exposure of the hydrogel to the thermal bonding temperatures does not have any apparent adverse effect on the gel. Moreover, one important advantage of the hydrogel valve is that when dry, it allows free passage of gases. In pneumatic systems, the dry hydrogel valve will allow the displacement of air from cavities and conduits upstream of an advancing liquid slug. Once the aqueous liquid arrives at the hydrogel's location, it will saturate and swell the gel, blocking the flow passage. Thus, the valve is self-actuated. The valve can be opened by heating the hydrogel to above its phase transition temperature. The hydrogel proved to be biocompatible in our testing and did not to hinder PCR. Moreover, the hydrogel valves did not appear to absorb significant quantities of DNA and enzymes suspended in PCR buffer.

Ice valves take advantage of the phase change of the working liquid itself—the freezing and melting of a portion of a liquid slug—to non-invasively close and open flow passages. An ice valve is electronically-addressable, does not require any moving parts, introduces only minimal dead volume, is leakage and contamination free, and is biocompatible. Moreover, in certain cases, the valve can operate in a self-actuated mode, alleviating the need for a sensor to determine the appropriate actuation time. For example, in a pneumatically driven system, the precooled conduit section would allow the free passage of air prior to the arrival of the liquid slug and would seal at the desired time when the slug arrives at the valve location.

Subsequent to their distribution into separate analysis paths, the various aliquots undergo a sequence of processing steps in reaction chambers. The reaction chambers are tailored to the nature of the target analyte. The analysis path for the detection of DNA will include the following main steps: pathogen lysis; DNA isolation and purification; PCR; isolation of the amplified DNA; mixing and incubation with target specific reporter particles; and capture of the labeled amplicon on a lateral flow strip. The analysis path for the detection of RNA comprises: cell lysis; RNA isolation and purification; Reverse Transcription PCR; isolation of the amplified DNA; mixing and incubation with target specific reporter particles; and capture of the labeled amplicons on a lateral flow strip. The analysis path for the detection of human antibodies to select pathogens comprises: dilution of sample; mixing and incubation with target specific reporter particles; capture on a lateral flow strip. The analysis path for the detection of pathogen antigens comprises dilution; solubilization or release of antigen; mixing and incubation with target specific reporter particles; and capture of labeled antigen on a lateral flow strip. Of course, the analysis paths described above focused on the lateral flow format. The invention also includes consecutive flow assays for the detection of antibodies. In the case of the consecutive flow assay, the analysis path will comprise: dilution, capture/enrichment of specific antibodies on a lateral flow strip; wash step to remove unbound antibodies; and detection by flowing reporter particles over the lateral flow strip.

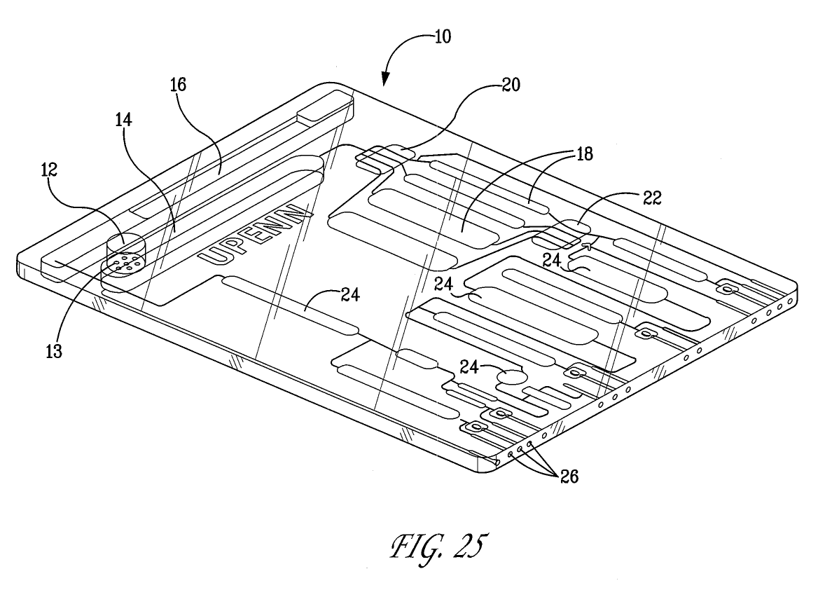

Turning to FIG. 25, an exemplary chip 10 is depicted. A sample inlet 12, having a rim 13, is disposed in the chip for receiving a sample. A dilution chamber 14 is disposed adjacent to the sample inlet 12 for adding a fluid to the sample. It is understood that a flow path exists between the sample inlet 12 and a detection zone 16. Although only one detection zone is depicted for simplicity, it is understood that there may be multiple detection zones.

A plurality of metering chambers 18 are disposed adjacent to the dilution chamber for precisely measuring the sample. The metering chambers 18 are controlled by an upstream valve 20 and a downstream valve 22.

A plurality of reaction chambers, generally given the reference numeral 24, are disposed adjacent to the metering chambers. Ports 26 are disposed in the chip 10 to supply reagents to the reaction chambers, or to provide propulsing fluids, or to remove excess fluids.

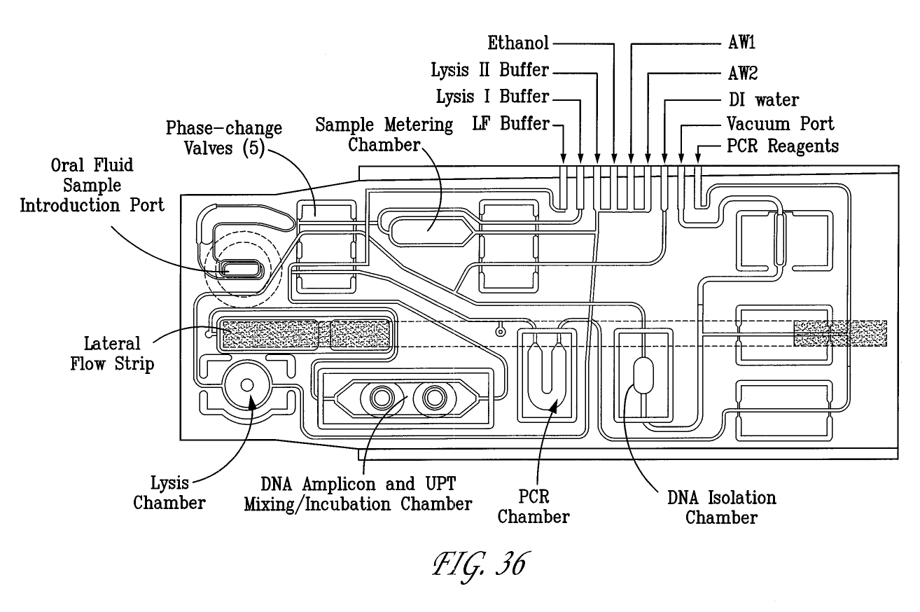

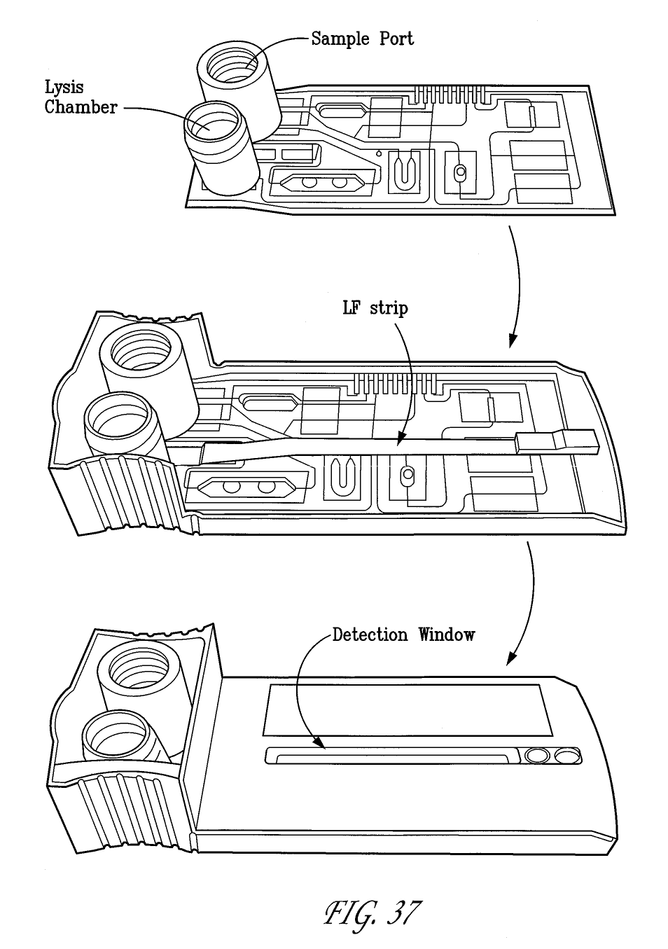

Referring now to FIG. 26, the depicted chip 10 enjoys many of the features of that of FIG. 25, but shows a cell lysis reaction chamber 24a, isolation reaction chamber 24b, PCR reaction chamber 24c, and a label incubation chamber 24d. Optional reagent storage chambers, generally given the reference numeral 30, are depicted for providing the desired reagents to the associated treatment chamber. A check valve 32 is depicted for allowing or preventing fluid flow. A solid support 34 is associated with the isolation reaction chamber 24b. Thus, the sample may be treated before introduction to the detection zone 16. A similar chip is depicted in FIGS. 36 and 37. It is understood that the chip may be disposed in a housing.

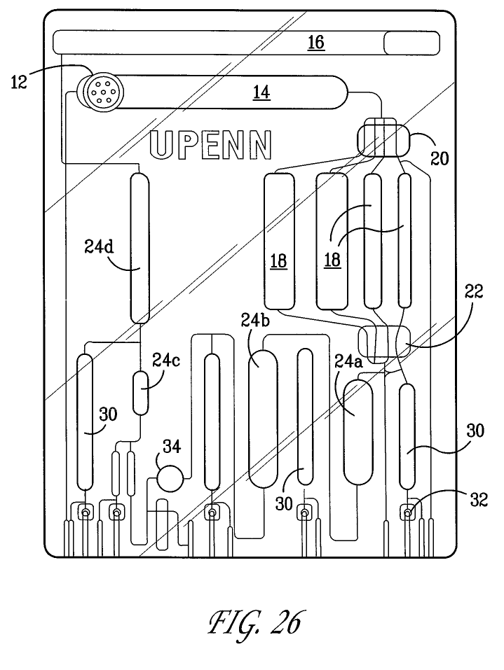

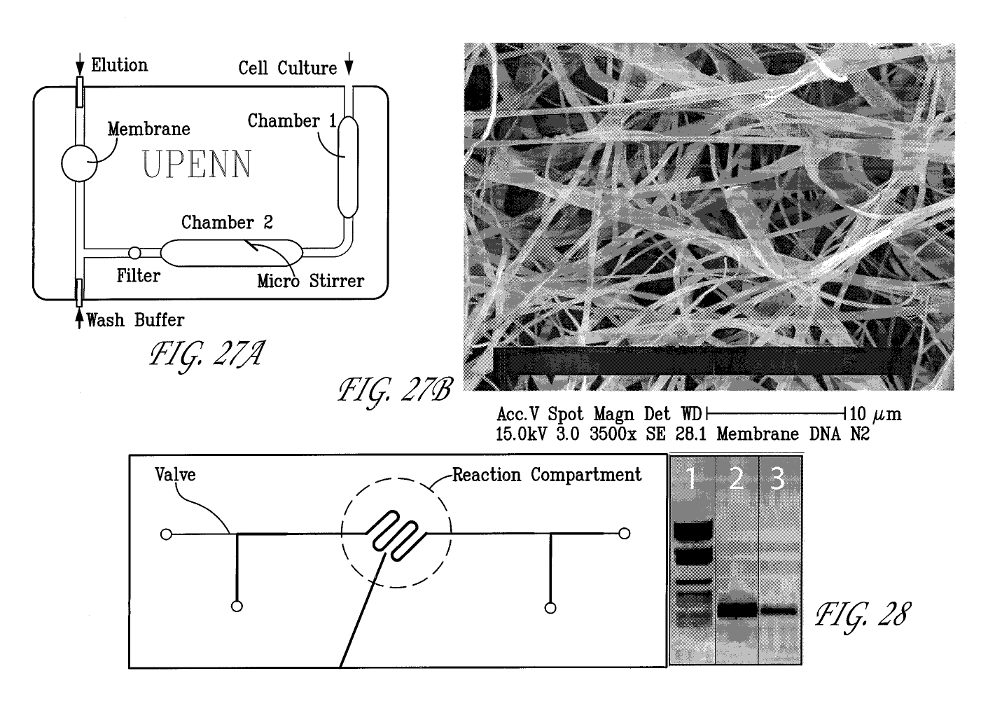

Referring now to FIGS. 25-28 the present invention also provides a chip, comprising a detection zone for interacting with either pre-selected RNA sequences or pre-selected DNA sequences and at least one further detection zone for interacting with pre-selected RNA sequences, DNA sequences, antibodies, or antigens.

In one embodiment, the first mentioned detection zone interacts with RNA and the at least one further detection zone interacts with DNA, antigen, or antibody. In another embodiment, the first mentioned detection zone interacts with DNA and the at least one further detection zone interacts with RNA, antigen, or antibody.

In one embodiment, the chip further comprises a plurality of detection zones wherein each detection zone independently interacts with RNA, DNA, antigen, or antibody.

While each detection zone does not have to be limited to a particular class of moiety, i.e., RNA, DNA, antigen, or antibody, it is understood that each detection zone can detect multiple examples within the moiety class if the detection zone if so treated. For example, the zones can interact with multiple antigens. In one embodiment, the first mentioned detection zone has a pre-selected pattern of zones, each for interacting with a different sequence. Likewise, in one embodiment, the further detection zone has a pre-selected pattern of zones, each for interacting with a different sequence of RNA, DNA, antigen, or antibody.

In one embodiment, the first mentioned detection zone is a chromatographic detection zone. In one embodiment, the detection zone is a polymeric material such as a nitrocellulose strip. The detection zone is contacted with capture sequences that are pre-selected for the pathogen. In some embodiments, multiple pathogens are tested for by providing complementary sequences pre-selected for the pathogens. Likewise, in one embodiment, the at least one further detection zone is a chromatographic detection zone. In one embodiment, the detection zone is a polymeric material such as a nitrocellulose strip. The detection zone is contacted with capture sequences that are pre-selected for the pathogen or compound of interest. In some embodiments, multiple pathogens are tested for by providing complementary sequences pre-selected for the pathogens.

It is understood that a sample lacking the pathogen(s) or compound(s) of interest will not interact with the detection zone. If present, the interaction between sample and sequence (s) is detectable. In one embodiment, the interaction is detectable through reporter particles.

As mentioned above, the chip includes a sample inlet for receiving a sample and a path between the sample inlet and the detection zone to allow fluid communication. In one embodiment, the chip further comprises a valve disposed in the path.

In one embodiment, the chip further comprises a port in fluid connection with the path for introducing reagents to the sample.

In one embodiment, the chip further comprises a port in fluid connection with the path for introducing a gas to move the sample through the path.

In one embodiment, the chip is disposable. In another embodiment, the chip is re-used. In another embodiment, the chip is archived.

The present invention provides a chip, comprising a sample inlet for receiving a sample; a detection zone in fluid communication with the sample inlet for interacting with either pre-selected RNA sequences, pre-selected DNA sequences, antigens, or antibodies from the sample; and a valve for controlling flow between the sample inlet and the detection zone.

In one embodiment, the chip further comprises a valve disposed in the path.

The chip may further comprise at least one further detection zone for interacting with pre-selected RNA sequences, DNA sequences, antibodies, or antigens from the sample.

In yet another embodiment of the present invention, a microfluidic chip is provided, comprising a PCR reaction chamber; and a phase change valve or a hydrogel valve for controlling the flow of a fluid into the reaction chamber.

When the reaction chamber is a PCR chamber, the format can be stationary (sample held in a chamber that is alternately heated and cooled, continuous flow through (sample propelled through a serpentine channel passing through a plurality of heating zones), pneumatic oscillatory (sample propelled back and forth through a conduit passing through a plurality of heating zones), self actuated (sample propelled through a closed loop containing a plurality of heating zones), electrokinetic (sample propelled by an electric field), or magneto-hydrodynamically (MHD)-driven (flow induced by electric current in the presence of a magnetic field).

One mode of achieving chip-based PCR is to hold the reagents in a chamber while cycling the chamber temperature (stationary PCR). One of the problems often experienced with stationary PCR microreactors is bubble formation. The bubbles are undesirable, as they may expel the reagents from the PCR chamber, thereby reducing the amplification efficiency. One way to minimize or eliminate the bubble formation is to pressurize the PCR chamber by sealing it.

The PCR mixture is driven into the reaction chamber through the inlet phase change (PC) valve. In one embodiment, effective mixing is realized by alternately propelling two fluids, for example, DNA elution and PCR reagents, into a chamber, thus significantly increasing the interface between the two fluids for better mixing. During this process, the inlet valve is maintained at room temperature, allowing unhindered passage of the liquid. The liquid fills the PCR chamber, displacing the air through the pre-cooled exit valve. Once the air has been displaced out of the chamber and the liquid arrives at the exit valve's location, it freezes and blocks the passage. Subsequently, the inlet PC valve is closed. Once both the upstream and downstream valves are closed, the temperature of the PCR reactor is cycled according to standard protocols. The subsequent increase in pressure suppresses bubble formation.

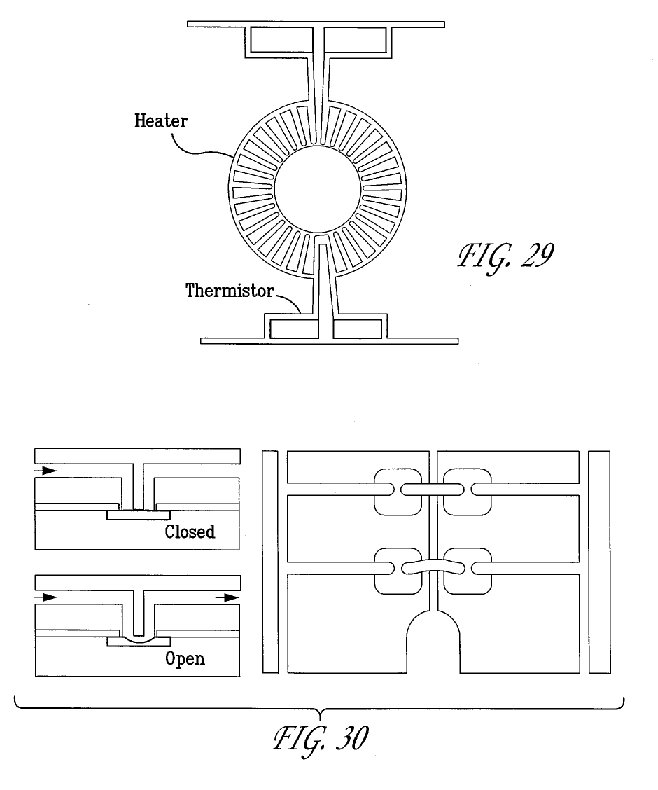

In operation, the chip receives a sample, which is treated as it moves through the chip, and then is applied to the detection zone. If the sample contains pathogens or antigens that the chip was pre-selected to detect (by placing the pre-selected RNA, DNA, antibodies, or antigens on the detection zone), an interaction will occur. The interaction can then be detected. FIG. 29 shows the various paths for DNA detection, antibody detection, antigen detection, and RNA detection, and the chip make-up depends upon the pre-selected analyte.

Referring to FIG. 30, a heater disposed on the chip is shown for heating the chambers.

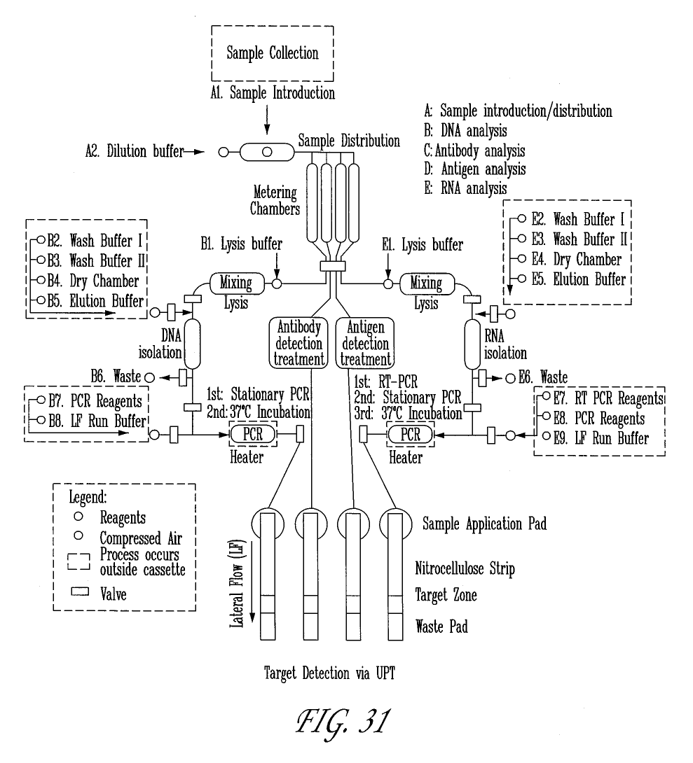

Referring to FIG. 31, a slab-based elasticity check valve is shown. In contrast to conventional flap-based design for check valve, the present valve design takes advantage of the elasticity of materials (e.g., PDMS) and use slab-based concept, significantly easing the fabrication and assembly. In the presence of sufficient pressure of the inlet flow, the valve opens; after the pressure is released, the valve closes. Figure A depicts the concept of the PDMS-based valve.

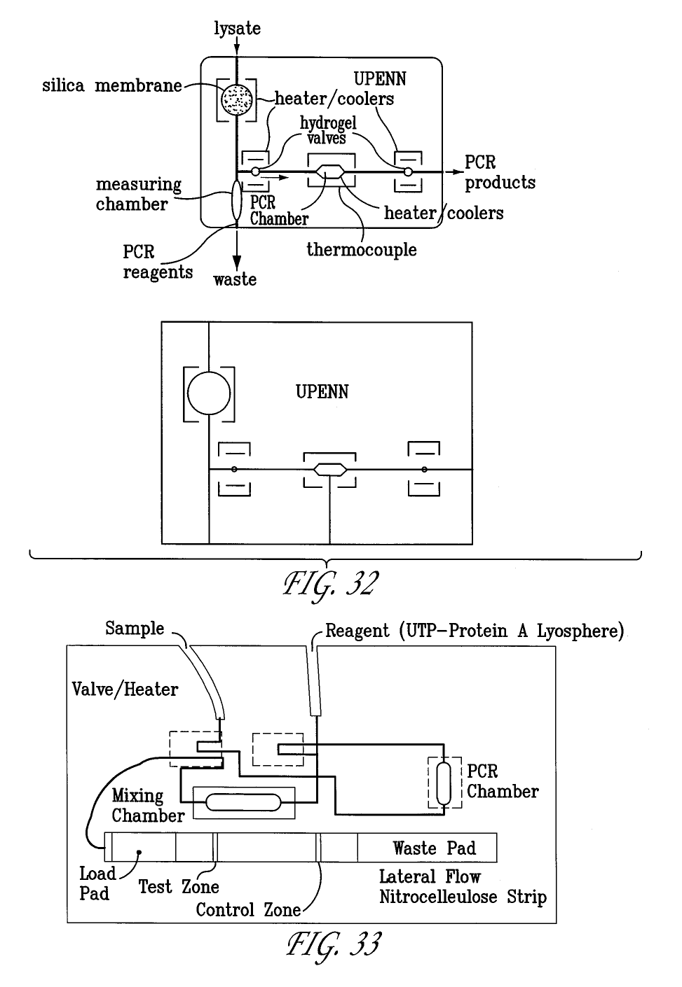

Referring to FIG. 32, a portion of a chip is shown. It is understood that the portion could function in a stand alone mode as a mini-chip, receiving cells, lysing them, isolating nucleotide sequences, then amplifying them via PCR. In one embodiment, lysis is performed in one chamber with optional venting. In one embodiment, lysis is performed as a two-step lysis at different temperatures, e.g., 37 C and 65 C for effectively lysing Gram-positive cells.

Referring to FIG. 33, a portion of a chip is shown. It is understood that the portion could function in a stand alone mode as a mini-chip, receiving purified nucleotides, amplifying them via PCR, and detecting pre-selected sequences.

The present invention relates, in part, to microfluidic systems, including valves and pumps for microfluidic systems. The valves of the invention include check valves, including diaphragm valves and flap valves. Other valves of the invention include one-use valves. The pumps of the present invention may include a reservoir and at least two check valves.

The present invention additionally relates to a method of making microfluidic systems including those of the present invention. The method includes forming a microfluidic system on a master, connecting a support to the microfluidic system and removing the microfluidic system from the master. The support may remain connected to the microfluidic system or the microfluidic system may be transferred to another substrate.

The present invention further relates to a method of manipulating a flow of a fluid in a microfluidic system. This method includes initiating fluid flow in a first direction and inhibiting fluid flow in a second direction and may be practiced with the valves of the present invention.

Traditionally, diaphragm-type microvalves have relied on a soft material (e.g., elastomer) for the diaphragm. Applicants have now recognized that it would be useful to develop a diaphragm in a non-elastomer material such as polycarbonate. Polycarbonate is inexpensive, and can be easily machined, injection molded, or hot embossed, as well as biochemically inert and biocompatible. It can also be thermally bonded to make laminated structures.

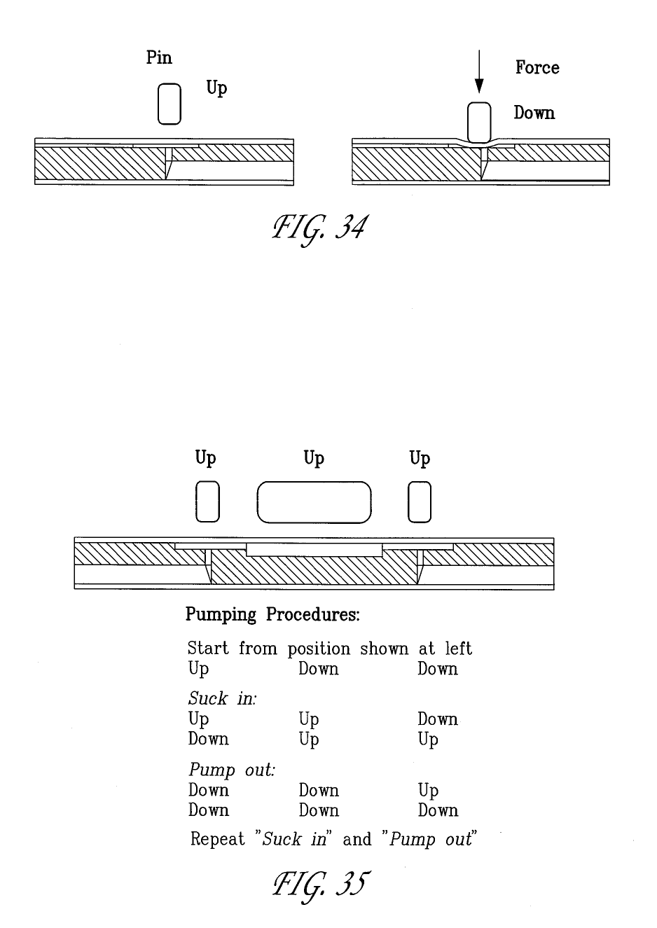

Referring to FIG. 34, a diaphragm-type microvalve is shown. The present invention teaches a method for using non-elastomeric materials for realizing diaphragm-type microvalves. The device design utilizes diaphragms made of thin layers of materials such as polycarbonate that are sufficiently deformable to deform, but not be elastic. An external force applied through an actuator, such as a pin or push rod, depresses the deformable member such that the flow path is narrowed or completely blocked. The actuator is moved by mechanical, electromechanical, magnetic, hydraulic, pneumatic, gravity, or centrifugal force; or volume change or phase change, or some combination thereof. Because the diaphragm can be constructed of the same material as that in which the microfluidic channels and chambers are defined, the fabrication and assembly are greatly simplified, compared to devices that use elastomer materials as the diaphragm. In this approach, one or more portions of one of the layers of the laminate microfluidic system can function as the diaphragms for one or more valves or pumps.

The deformable member may be the same material as the material hosting the channel under control of the valve. As an example, a flow path is defined as a 0.25-mm wide, in a 2-mm polycarbonate laminate structure that serves as a substrate in which a microfluidic circuit is formed. In this example, there is seat that receives the membrane. An orifice in the seat connects the two channels. A thin (0.25-mm) sheet of polycarbonate is thermally bonded to the substrate. An external force is locally applied to the deformable membrane, such that the membrane contact the seat, thus constricting or blocking the passage for flow. In one embodiment, the deformable member has a thickness from about 10 μm to about 1000 μm. In one embodiment, the deformable member has a thickness of about 250 μm.

Turning to FIG. 35, a micropump schematic is provided. A pair of valves such as those described with reference to FIG. 34 can be used, having a pumping chamber disposed between them, and an actuator for pressing on the deformable member adjacent to the pumping chamber. By selectively applying the respective actuators, the micropump can move fluid as described in the schematic.

### EXAMPLES AND OTHER ILLUSTRATIVE EMBODIMENTS

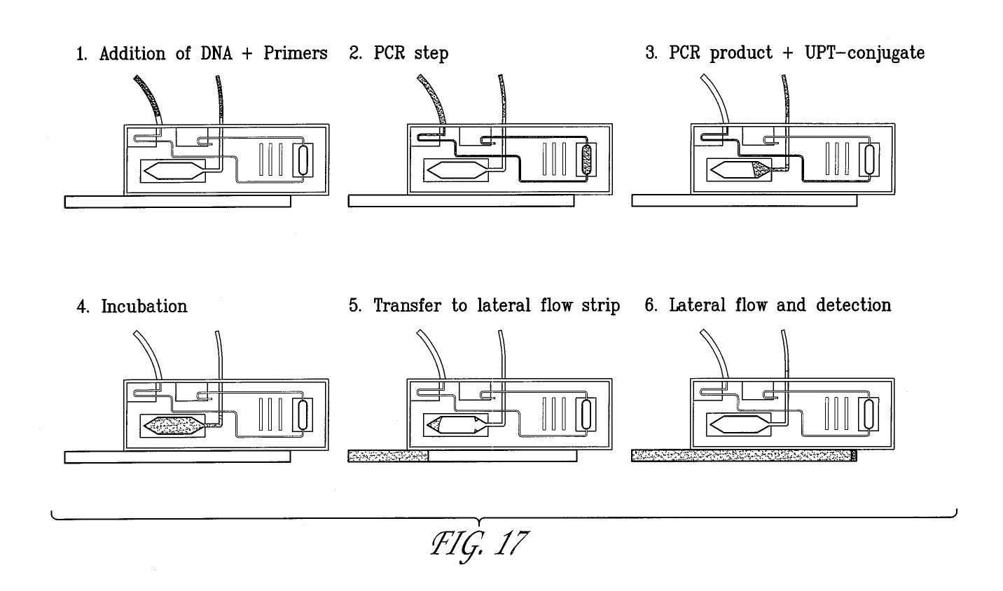

A cassette was designed, constructed, and successfully tested to carry out PCR and to detect the amplified DNA (FIG. 15). FIG. 16 shows a schematic of a partially integrated PCR and Detection cassette. This is the downstream component of the analysis path for pathogen RNA or DNA. The device demonstrates the partial integration of the PCR chamber, a mixing and incubation (37° C.) chamber, and a detection compartment. Two phase-change valves were used to assist in the PCR chamber filling and sealing. The downstream valve was pre-cooled. When the PCR mixture arrived at the valve location, it froze and blocked the passage. Once the PCR chamber was filled, the upstream valve was closed. The PCR thermocycling was achieved with a thermoelectric module.

At the completion of the amplification process the PCR products were propelled to the mixing chamber where they mixed with buffer solution containing UPT particles for detection. Mixing was accomplished by cooling and heating the mixing chamber with two thermoelectric modules. After incubation at 37° C. for 30 min, the mixture was pneumatically propelled into the loading pad of the detection strip. The solution was drawn into the strip by capillary forces and the presence of the UPT particles was detected by exciting the UPT particles and scanning the emitted signal. The control algorithms for the fluid flow, heating, and cooling were implemented in LabVIEW™.

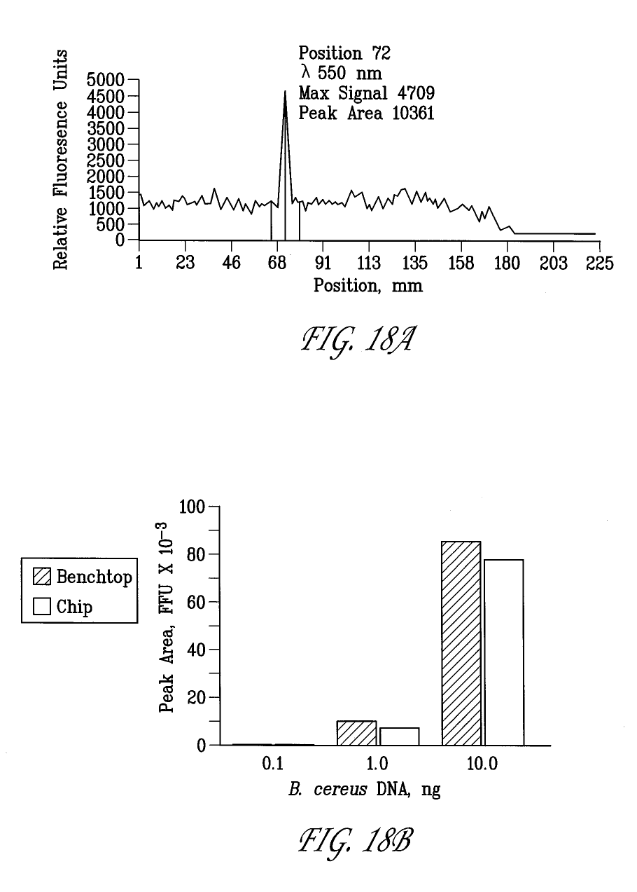

FIG. 17 shows images taken during treatment.

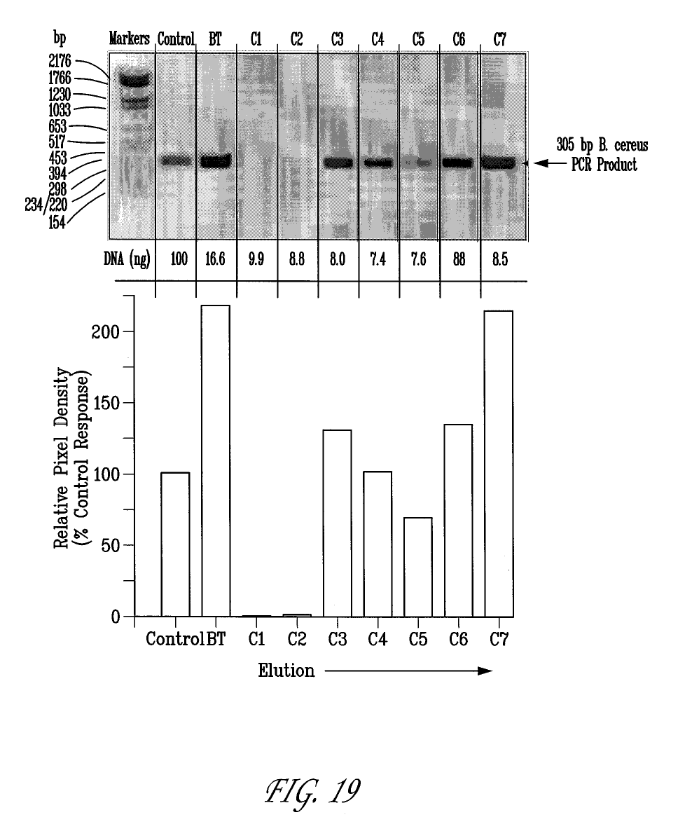

FIG. 18 shows the comparable results of detection between benchtop and cassette based runs.

FIG. 19 shows PCR amplification of B. cereus DNA obtained by benchtop vs. cassette lysis and isolation. B. cereus (2×109 cell/ml) was lysed either by conventional benchtop methods (BT) or in the cassette (FIG. 13A). Purified DNA was eluted in 7 fractions and they were used as templates for PCR. (A) shows the agarose gel results comparing cassette lysed/purified DNA to whole genomic DNA (control), BT (benchtop lysed/purified DNA and cassette lysed/purified fractions. B. Relative pixel density of the PCR product from each fraction. Quantization of the gel image captured with a Kodak Image station used ImageQuant V5.2 software.

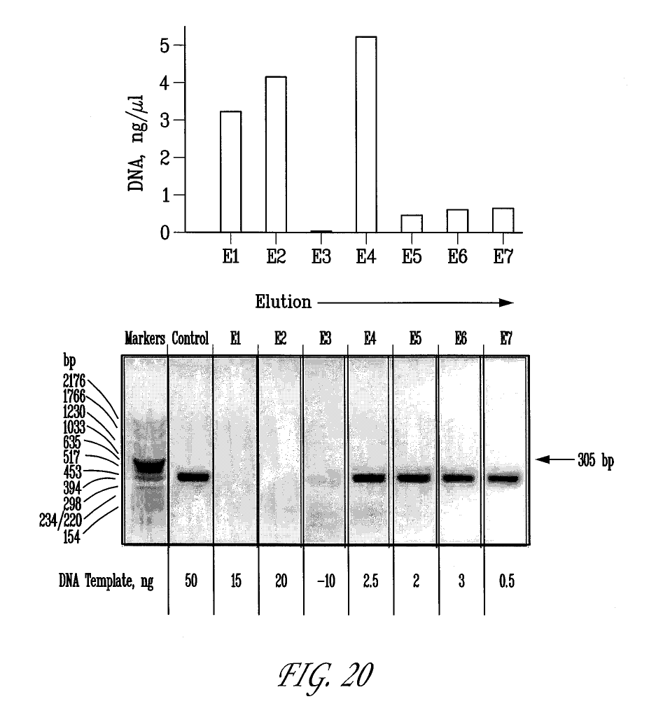

FIG. 20 is an agarose gel image of cassette isolated B. cereus DNA PCR products from a partially integrated DNA isolation and PCR device. PCR was performed for 25 cycles producing the anticipated 305 bp B. cereus amplicon.

The disclosures of each patent, patent application, and publication cited or described in this document, if any, are hereby incorporated herein by reference in their entireties.

Various modifications of the invention, in addition to those described herein, will be apparent to those skilled in the art from the foregoing description. Such modifications are also intended to fall within the scope of the appended claims.

The disclosures of each patent, patent application, and publication cited or described in this document, if any, are hereby incorporated herein by reference in their entireties.

Various modifications of the invention, in addition to those described herein, will be apparent to those skilled in the art from the foregoing description. Such modifications are also intended to fall within the scope of the appended claims.

1. A system, comprising:
a cassette having at least one port and a sample inlet in fluid communication with a detection zone capable of interacting with pre-selected RNA sequences, DNA sequences, antibodies, antigens, or mixtures thereof, if present, in a sample; and
a developer for engaging the port of the cassette, wherein the developer is capable of propelling the sample from said sample inlet to said detection zone.

2. The system of claim 1, wherein the propulsion is hydraulic, pneumatic, electric, or magnetic, or mixtures thereof.

3. The system of claim 1, wherein the cassette further comprises at least one further detection zone for interacting with RNA, DNA, or antigen.

4. The system of claim 1, wherein the cassette includes a valve for controlling flow between the sample inlet and the detection zone.

5. The system of claim 4, wherein the developer has means for controlling the valve.

6. The system of claim 1, wherein the developer includes a pump.

7. The system of claim 1, wherein the developer includes a heater/cooler.

8. The system of claim 6, wherein the heater/cooler is a Peltier heater/cooler.

9. The system of claim 1, wherein the developer dispenses a reagent.

10. The system of claim 1, wherein the developer dispenses a buffer to a cassette having a pre-loaded reagent.

11. The system of claim 1, further comprising a treating reagent.

12. The system of claim 11, wherein the treating reagent is directed to RNA isolation and amplification.

13. The system of claim 11, wherein the treating reagent is directed to DNA isolation and amplification.

14. The system of claim 11, wherein the treating reagent is directed to antibody detection.

15. The system of claim 11, wherein the treating reagent is directed to antigen detection.

16. The system of claim 11, wherein the reagent is for labeling the interacted RNA, DNA, antibody, or antigen.

17. The system of claim 16, wherein the label is a reporter particle.

18. The system of claim 16, further comprising a detector for detecting the labeled RNA, DNA, antibody, or antigen.

19. The system of claim 18, wherein the detector is a UPT detector.

20. A system, comprising:
a cassette having at least one port and a sample inlet in fluid communication with a detection zone for interacting with pre-selected RNA sequences, DNA sequences, antibodies, antigens, or mixtures thereof, if present, in a sample;
a developer for engaging the port of the cassette, wherein the developer propels the sample from said inlet to said detection zone; and
a detector for detecting the pre-selected RNA sequences, DNA sequences, antibodies, antigens, or mixtures thereof.

21. The system of claim 20, wherein the cassette includes a valve for controlling flow between the sample inlet and the detection zone.

22. The system of claim 20, wherein the developer has means for controlling the valve.

23. The system of claim 20, wherein the cassette has an identifier which provides information to the developer.

24. The system of claim 20, wherein the developer supplies reagents for developing the cassette.

25. The system of claim 20, wherein the reagents for developing the cassette are stored on the cassette.

26. The system of claim 20, wherein the developer supplies propulsion for the microfluidics.

27. The system of claim 20, wherein the cassette supplies at least a portion of the propulsion for the microfluidics.

28. The system of claim 20, comprising a plurality of cassettes adapted to detect pre-selected disorders or analytes.

29. The system of claim 20, wherein each cassette is disorder or analyte specific, whereas the developer is adapted to develop any of the cassettes.

30. The system of claim 29, wherein the developer contains a plurality of reagents, at least a portion of the reagents are capable of developing a specific cassette.

31. The system of claim 20, further comprising a quick connect system between the developer and cassette.

32. A method for concurrent testing for at least two of RNA, DNA, antibody, and antigen in a sample, comprising:
applying a portion of the sample to a detection zone disposed on a microfluidic cassette for interacting with pre-selected RNA sequences, DNA sequences, antibodies, or antigens, or mixtures thereof; and
applying at least one further portion of the sample to at least one further detection zone disposed on the microfluidic cassette for interacting with pre-selected RNA sequences, DNA sequences, or antigens.

33. The method of claim 32, further comprising applying a portion of the sample to another detection zone, wherein the detection zone interacts with RNA, DNA, antigen, or antibody.

35. The method of claim 32, further comprising detecting the interaction.

35. The method of claim 35, wherein interaction is detected using UPT particles, fluorescing particles, hybridization sensors, or electrochemical sensors.

36. A method for testing for the presence of a pre-selected pathogen in a sample, comprising:
placing the sample in a microfluidic cassette;
metering the sample;
propelling the sample along a flow path in the cassette to a detection zone having at least one zone adapted to interact with the pre-selected pathogen; and
detecting the presence or absence of interaction.

37. The method of claim 36, wherein there is a pre-selected pattern of zones on the detection zone, each for interacting with a different sequence.

38. The method of claim 36, further comprising applying a portion of the sample to a pre-selected pattern of zones on at least one further detection zone, each zone for interacting with a different sequence of RNA, DNA, antigen, or antibody.

39. A method of testing for pre-selected pathogens, comprising:
placing a sample in a cassette; and
propelling the sample through the cassette under pressure, wherein a portion of the sample is directed to a detection zone for interacting with pre-selected RNA sequences, DNA sequences, antibodies, or antigens known to be associated with a pre-selected pathogen.

40. The method of claim 39, wherein the propulsion is hydraulic, electric, or magnetic.

41. The method of claim 39, further comprising controlling movement of the sample with a valve disposed in the cassette.

42. The method of claim 39, further comprising diluting the sample.

43. The method of claim 39, wherein at least one reagent is pre-loaded.

44. The method of claim 39, further comprising metering the sample.

45. The method of claim 39, further comprising treating the sample.

46. The method of claim 45, further comprising lysing cells in the sample.

47. The method of claim 45, further comprising isolating RNA or DNA in the sample.

48. The method of claim 47, wherein the RNA or DNA are bound to a solid phase.

49. The method of claim 47, further comprising amplifying RNA or DNA in the sample.

50. The method of claim 49, the cassette further comprising a PCR chamber in fluid communication the detection zone, wherein the RNA or DNA is amplified using PCR in the PCR chamber.

51. The method of claim 50, wherein the PCR chamber is pressurized to suppress bubble formation.

52. The method of claim 39, further comprising detecting the interaction by attachment of a reporter particle.

53. A method for testing for HIV in a sample, comprising:
providing a microfluidic cassette having means for testing for RNA sequences associated with HIV and means for testing for antigens associated with HIV.

54. A method for filling and emptying of a closed loop, comprising:
providing an ice valve in the loop between an inlet and outlet;
closing the valve to fill the loop;
opening the valve to circulate fluid; and
closing the valve to empty the loop out the outlet.

55. A method for mixing fluids in a chamber without bubble formation, comprising:
adding a fluid;
freezing the fluid;
adding at least one further fluid; and
thawing the first fluid.

56. A method for performing PCR in a chamber without bubble formation, comprising:
providing a valve at each inlet and outlet of the chamber; and
closing the valves.

57. A microfluidic chip, comprising:
a first detection zone for interacting at least a portion of a sample with pre-selected RNA sequences, DNA sequences, antibodies, or antigens, or any combination thereof;
at least one further detection zone for interacting with pre-selected RNA sequences, DNA sequences, antigens, or any combination thereof,
wherein at least one of the detection zones comprises a chromatographic material comprising a polymeric material, an array of pillars, grooves, a lateral flow membrane, or any combination thereof; and
at least one flow path for contacting each of the detection zones with the sample or portion thereof.

58. The chip of claim 57, wherein said pre-selected sequences, antibodies, or antigens are those associated with at least one known pathogen, disorder, or for a pre-selected gene, or for a contaminant.

59. The chip of claim 57, further comprising a plurality of detection zones, wherein each detection zone is capable of independently interacting with RNA, DNA, an antigen, an antibody, or any combination thereof.

60. The chip of claim 57, wherein at least one of the detection zones comprises a pre-selected pattern of zones, each capable of interacting with a different sequence of RNA, DNA, antigen, or antibody.

61. The chip of claim 57, wherein the interaction is detectable through reporter particles.

62. The chip of claim 61, wherein the reporter particles comprise phosphor particles, fluorescing particles, hybridization sensors, particle arrays, electrochemical sensors, or any combination thereof.

63. A microfluidic chip, comprising:
a sample inlet for receiving a sample and a path between the sample inlet and the detection zone to allow fluid communication, the path comprising a valve disposed in the path;
a first detection zone for interacting with either pre-selected RNA sequences or pre-selected DNA sequences; and
at least one further detection zone for interacting at least a portion of the sample with pre-selected RNA sequences, DNA sequences, antibodies, or antigens.

64. The chip of claim 63, wherein the first mentioned detection zone interacts with RNA and the at least one further detection zone interacts with DNA, antigen, or antibody.

65. The chip of claim 63, wherein the first mentioned detection zone interacts with DNA and the at least one further detection zone interacts with RNA, antigen, or antibody.

66. The chip of claim 63, further comprising a plurality of detection zones, wherein each detection zone independently interacts with RNA, DNA, antigen, or antibody.

67. The chip of claim 63, wherein the first mentioned detection zone has a pre-selected pattern of zones, each for interacting with a different sequence.

68. The chip of claim 63, wherein the further detection zone has a pre-selected pattern of zones, each for interacting with a different sequence of RNA, DNA, antigen, or antibody.

69. The chip of claim 63, wherein the valve is a phase change valve, a hydrogel valve, or a mechanical valve.

70. The chip of claim 63, further comprising a chamber disposed in the path for metering the sample.

71. The chip of claim 63, further comprising a port in fluid connection with the path for introducing reagents to the sample.

72. The chip of claim 63, further comprising a port in fluid connection with the path for introducing a gas to move the sample through the path.

73. The chip of claim 63, further comprising a chamber for treating the sample.

74. The chip of claim 73, wherein the treating chamber is a cell lysis chamber, a nucleic acid entrainment chamber, a PCR chamber, or a label incubation chamber.

75. The chip of claim 63, further comprising a reagent chamber preloaded with reagent.

76. The chip of claim 63, further comprising a waste chamber.

77. A microfluidic chip, comprising:
a detection zone for interacting with pre-selected RNA sequences, DNA sequences, antibodies, or antigens, or mixtures thereof;
at least one further detection zone for interacting pre-selected RNA sequences, DNA sequences, antibodies, or antigens;
wherein when the first detection zone is selected to interact with DNA sequences, the at least one further detection zone interacts with pre-selected RNA sequences, antibodies, or antigens, and wherein when the first detection zone is selected to interact with antigens, the at least one further detection zone interacts with pre-selected RNA sequences, DNA sequences, or antibodies; and
at least one flow path for contacting the detection zones with a sample.

78. The chip of claim 77, further comprising chambers for at least one of cell and virus lysis, nucleic acid isolation, nucleic acid amplification, and the labeling nucleic acids, antigens, or antibodies.

79. The chip of claim 77, wherein the detection zone is a lateral flow strip with capture zones that selectively bind analytes of interest, rendering them detectable.

80. The chip of claim 77, wherein labeled nucleic acids are blotted onto a lateral flow strip to initiate capillary flow of nucleic acids along said strip, resulting in their capture at zones formed in pre-selected areas of the strip.

81. A microfluidic chip, comprising:
a diaphragm valve, comprising
an actuator; and
a deformable member, wherein the deformable member is a non-elastomer and
has a thickness in the range of from about 10 μm to about 1000 μm; and
a micropump, comprising
a pumping chamber disposed between a pair of diaphragm valves;
a deformable member adjacent to the pumping chamber; and
an actuator for pressing on the deformable member.
